(19) United States
(12) Patent Application Publication
(10) Pub. No.: US 2024/0298678 A1
(43) Pub. Date: Sep. 12, 2024

(54) GROWTH FACTORS FOR LABORATORY GROWN MEAT AND OTHER APPLICATIONS

(71) Applicant: Terasaki Institute for Biomedical Innovation, Los Angeles, CA (US)

(72) Inventors: Seyedvahid Hosseini, Newbury Park, CA (US); Alireza Khademhosseini, Los Angeles, CA (US)

(73) Assignee: Terasaki Institute for Biomedical Innovation, Los Angeles, CA (US)

(21) Appl. No.: 18/281,033

(22) PCT Filed: Mar. 9, 2022
(86) PCT No.: PCT/US2022/019628

§ 371 Date: Sep. 8, 2023

Related U.S. Application Data
(60) Provisional application No. 63/314,171, filed on Feb. 25, 2022.
(60) Provisional application No. 63/314,191, filed on Feb. 25, 2022.
(60) Provisional application No. 63/300,577, filed on Jan. 18, 2022.
(60) Provisional application No. 63/279,617, filed on Nov. 15, 2021.
(60) Provisional application No. 63/279,631, filed on Nov. 15, 2021.
(60) Provisional application No. 63/279,642, filed on Nov. 15, 2021.
(60) Provisional application No. 63/279,644, filed on Nov. 15, 2021.
(60) Provisional application No. 63/164,397, filed on Mar. 22, 2021.
(60) Provisional application No. 63/164,387, filed on Mar. 22, 2021.
(60) Provisional application No. 63/159,403, filed on Mar. 10, 2021.

(51) Int. Cl. A23L 13/00; C12N 5/077

(52) U.S. Cl. CPC A23L 13/00; C12N 5/0658; C12N 2500/84; C12N 2501/105; C12N 2501/11; C12N 2501/115; C12N 2501/15; C12N 2533/56

(57) ABSTRACT

The present disclosure generally relates to an improved cell-culture growth medium, for example, for the production of cell-based meat, and other cultivated products, or applications. For example, certain embodiments are directed to a cell culture growth factor supplement comprising platelet lysate (PL) and platelet rich plasma (PRP). Such solutions may be used, for example, to increase the cellular biomass in a bioreactor by enhancing the rate and frequency of cellular proliferation. In some embodiments, platelet-rich plasma is isolated from the whole blood of a living animal. The platelets within the platelet-rich plasma may be concentrated, for example, by centrifugation, to produce a platelet concentrate. In some embodiments, the platelet concentrate can be cultivated and activated using an agonist to release growth factors. In some embodiments, the growth factor solution can be separated from the platelets and added to a bioreactor containing a cell-based meat product.

### RELATED APPLICATIONS

This application is a national stage filing under 35 U.S.C. § 371 of International Patent Application Serial No. PCT/US2022/019628, filed Mar. 9, 2022, entitled “Growth Factors for Laboratory Grown Meat and Other Applications,” which claims the benefit of U.S. Provisional Patent Application Ser. No. 63/159,403, filed Mar. 10, 2021, entitled “Constructs for Meat Cultivation and Other Applications”; U.S. Provisional Patent Application Ser. No. 63/279,617, filed Nov. 15, 2021, entitled “Constructs Comprising Fibrin or Other Blood Products for Meat Cultivation and Other Applications”; U.S. Provisional Patent Application Ser. No. 63/279,631, filed Nov. 15, 2021, entitled “Methods and Systems of Preparing Cultivated Meat from Blood or Cellular Biomass”; U.S. Provisional Patent Application Ser. No. 63/279,642, filed Nov. 15, 2021, entitled “Systems and Methods of Producing Fat Tissue for Cell-Based Meat Products”; U.S. Provisional Patent Application Ser. No. 63/279,644, filed Nov. 15, 2021, entitled “Production of Heme for Cell-Based Meat Products”; U.S. Provisional Patent Application Ser. No. 63/300,577, filed Jan. 18, 2022, entitled “Animal-Derived Antimicrobial Systems and Methods”; U.S. Provisional Patent Application Ser. No. 63/164,397, filed Mar. 22, 2021, entitled “Growth Factor for Laboratory Grown Meat”; U.S. Provisional Patent Application Ser. No. 63/164,387, filed Mar. 22, 2021, entitled “Methods of Producing Animal Derived Products”; U.S. Provisional Patent Application Ser. No. 63/314,171, filed Feb. 25, 2022, entitled “Growth Factors for Laboratory Grown Meat and Other Applications”; and US Provisional Patent Application Ser. No. 63/314,191, filed Feb. 25, 2022, entitled “Methods and Systems of Producing Products Such as Animal Derived Products.” Each of these is incorporated herein by reference in its entirety.

### FIELD

The present disclosure relates to an improved cell-culture growth medium for the production of cultured animal derived products including cultivated meat.

### BACKGROUND

Cultivated meat, or cell-based meat, is meat that is produced using in vitro cell culture or bioreactors, instead of being harvested from live animals. In many cases, the meat that is produced may include muscle cells and fat cells. Such meat may include, for example, chicken, beef, pork, or fish. Such technologies have the potential to revolutionize agriculture, for example, by decreasing the amount of land necessary to produce meat, avoiding unethical farming of animals, or increasing the available food supply. However, cultivated meats are difficult and expensive to manufacture, and thus improvements are needed.

Cell-based cultivated meat is a recent innovation in the food industry. Cell-based meat can be manufactured by culturing non-human animal cells in vitro to create a meat without farming animals in the traditional way. As used herein cell-based meat is synonymous with cultivated meat, cultured meat, cellular meat, slaughter-free meat, and synthetic meat, among other related terms.

A significant amount of research has been done on producing a variety of cell-based meats from different spices of animals including mammals, fishes and insects. It is projected that by 2030, the world population shall have surpassed 8.5 billion people. This projection will require a surplus of food production to suffice. The United States Department of Agriculture has estimated that 450 million tons of animal waste is produced annually.

In addition, global production and consumption of meat continue to surge as demand is driven upward by population growth, individual economic gain, and urbanization. In 2012, the Food and Agriculture Organization (FAO) of the United Nations projected the global demand for meat would reach 455M metric tons by 2050 (a 76% increase from 2005). Likewise, the global demand for fish is projected to reach 140M metric tons by 2050.

This rising demand is problematic as current methods of large-scale animal husbandry are linked to public health complications, environmental degradation and animal welfare concerns. With regard to human health, the animal agriculture industry is interconnected with foodborne illness, diet-related disease, antibiotic resistance, and infectious disease. Notably, zoonotic diseases (e.g., Nipah virus, influenza A) are linked to agricultural intensification. Animal agriculture also contributes to environmental issues including greenhouse gas emissions, land use, and water use. The United Nations Intergovernmental Panel on Climate Change released a 2018 report asserting that greenhouse gas emissions must be reduced 45% by 2030 to prevent global temperatures from increasing 1.5° C.; a target that could mitigate catastrophes associated with a 2.0° C. increase. Conventional mitigation techniques include improvements in reforestation, soil conservation, waste management as well as tax policy, subsidies, and zoning regulations. While these strategies remain important, the urgency of climate change may require more transformative approaches. Lastly, with regard to animal welfare concerns, each year billions of animals are killed or suffer either directly (e.g., farm animal slaughter, seafood fishing) or indirectly (e.g., fishing by-catch, wildlife declines due to habitat destruction) in relation to human food systems.

The majority of the aforementioned issues can be attributed to the fact that the raw material inputs (i.e., animals) for conventional meat production are inherently unsanitary, inefficient, and sentient. By removing animals from the manufacturing process, several externalities may be alleviated.

Cultivated meat or cell-based meat is an alternative source of meat to replace animal-based meat. Cell-based meat is projected to be common in the global market in a few years, although one of the major challenges is the high cost associated with production of cultivated meat. Unfortunately, the economics of cell-based meat production are problematic with respect to large scale commercialization. The cultured beef burger cultivated by Maastricht University in 2013 is reported to have cost $280,400 ($2,470,000/kg) to produce. The production process involved three researchers using bench-scale techniques to expand 20,000 muscle cells over three months and served as a proof-of-concept rather than an attempt to scale production. A few groups have performed preliminary economic analyses to project the cost of cell-based meat for large-scale production scenarios. In 2008, The In Vitro Meat Consortium estimated, by modeling capital and growth medium costs based on data for single-cell protein production, cell-based meat could cost approximately twice as much as chicken. In 2014, a study speculating on the technical, societal, and economic factors of village-scale cell-based meat production calculated a cost range of $11-520/kg dependent on the price of growth medium. Selected companies are targeting high-value products (e.g., foie gras, bluefin tuna, kangaroo meat) in order to lower the bar for reaching price parity.

One challenge to the economics of cell-based meat production relates to the price and availability of suitable growth media for such production. Currently, Fetal Bovine Serum (FBS), which comes from the fetus of a bovine, is one of the main growth factor supplements used for cell culture. Obtaining FBS is impractical in the large quantities needed for large scale production of cultivated meat, since it involves the slaughter of pregnant cows and results in more slaughtered animals than conventional meat production. Since FBS remains one of the preferred media for cell-based meat production, there exists a need for a sustainable and retainable replacement in order to bring cell-based meat to the market.

Fully defined media, such as Essential 8™, which were developed for stem cell culture and for human therapeutic proposes, are not suitable for the cultivation of myoblasts, fibroblasts and adipocyte, which are often essential for production of cultivated meat. Moreover, the need to supplement such solutions with growth factors, obtained as freeze-dried powders, for example from commercial vendors, is highly expensive, and may impose a huge production cost to manufacture at the scale needed for the cultivated meat.

In a recent attempt, Good Food Institute developed a more realistic calculation for the cost of producing cultivated meat using a growth medium based on Essential 8™ (ThermoFisher Scientific). According to that calculation, the cost of growth media represented the most significant expense for such production, even when produced at large scale and at a food grade rather than pharmaceutical grade specification.

Of interest to the present disclosure is the proposal to use human platelet lysate (hPL) as a replacement for FBS for use in human cell culture for variety of biomedical applications, such as stem cell therapy and tissue engineering as an animal free growth factor to avoid regulatory hindrance associated with the use of non-human animal products. hPL has shown to be useful as a supplement for growth of many types of cells, particularly stem cells. hPL is generally a byproduct of platelet donation for other therapeutic proposes. Usually, the donated platelet has an expiration date of a week and after that is frozen and with thawing it is called platelet lysate. Since the platelets are heavily loaded with variety of growth factors, they release growth factors useful for growing cells after releasing from platelet during thawing process. In fact, it is a primary biological function of platelets that they release growth factors at the site of injury to promote healing of injured tissues. Despite the use of human platelet lysate for therapeutic applications such as wound healing, or to aid healing of other injuries such as to tendons and cartilage and culture of human cells for therapeutic application, animal platelet lysate is less used for therapeutic purposes. For example, horse PL can be extracted and injected into animal joints to promote the healing of the injured joint or ligament.

Of interest is the disclosure of Dietz, US Patent Publication 2011/0171731 which discloses methods and materials for using platelet lysate compositions to grow stem cells. Dietz, US Patent Publication 2015/0329820 discloses cell culture media compositions for growing mammalian cells including a human platelet lysate comprising a lysed human apheresis platelet preparation.

Animal derived platelet lysate (PL) or platelet rich plasma (PRP) from different animals such as bovine, equine and canine have been used to treat a wide range of injuries in veterinary medicine.

Of interest is the disclosure of Strunk, US Patent Publication 2012/0276632, which proposes the use of lysates of human platelet-rich plasma (PRP) such as obtained by apheresis as a substitute for FBS in human cell culture media.

Fetal Bovine Serum (FBS) is generally preferred by those of ordinary skill in the art as a growth medium for cell cultures because it is so rich in nutrients, but it is relatively expensive and is limited in its supply. Accordingly, there remains a need in the art for improved growth media and methods for its production for cultured animal derived products including cell-based meat production.

### SUMMARY

The present disclosure relates to an improved cell-culture medium for the production of cultured animal derived products including cultivated meat. The subject matter of the present disclosure involves, in some cases, interrelated products, alternative solutions to a particular problem, and/or a plurality of different uses of one or more systems and/or articles.

The improved media, in certain embodiments, comprises the combination of platelet lysate (PL) with platelet rich plasma (PRP) which is not only inexpensive but is equivalent to fetal bovine serum (FBS) in promoting cell culture growth. Provided in certain cases are compositions comprising platelet lysate (PL) with platelet rich plasma (PRP), optionally comprising other nutrient ingredients. In some embodiments, the concentration of platelet lysate (PL) plus platelet rich plasma (PRP) comprises between 2 to 20 weight percent of the entire composition.

In some embodiments, the concentration of platelet lysate plus platelet rich plasma comprises between 5 to 15 weight percent of the entire composition. In some embodiments, the concentration of platelet lysate plus platelet rich plasma comprises about 10 weight percent of the entire composition. The concentration of platelet lysate and/or platelet rich plasma in the cell culture growth media can be measured using any technique known by those skilled in the art. For example, the total concentration of growth factors in such solutions can be determined using spectrophotometry at 280 nm. In some embodiments, the concentration of platelets and platelet components in the cell culture media are between 2 mg/mL and 20 mg/mL. In some embodiments, the concentration of platelets and platelet components in the cell culture media are between 9 mg/mL and 11 mg/mL.

While PL and PRP can be obtained from the blood and blood products of slaughtered livestock, in some embodiments, the PL+PRP combination can be sustainably sourced from live animals to produce cultured animal derived products according to the methods of U.S. Ser. No. 63/164,387 filed Mar. 22, 2021, entitled “Methods of Producing Animal Derived Products,” the disclosure of which is hereby incorporated by reference.

In some embodiments, the cell culture growth media comprising platelet lysate (PL) and platelet rich plasma (PRP) can include those components obtained from the same or multiple vertebrate or invertebrate species. Exemplary species include but are not limited to a variety of mammalian, avian, reptilian, amphibian, avian and fish species including but not limited to those selected from the group consisting of cow, sheep, goat, swine, deer, camel, whale, fowl, fishes, crabs, and shrimps.

According to one aspect of the disclosure, the cell culture growth media comprises a platelet lysate comprising a platelet lysate (PL) and a platelet rich plasma comprising platelet rich plasma (PRP).

In some embodiments, the platelet lysate (PL) and platelet rich plasma (PRP) can be obtained in a variety of manners, including from animals slaughtered for food, but these can also be harvested from living animals raised for the purpose of providing such materials in a sustainable manner not unlike the use of dairy cows to produce milk. For example, the components can be harvested from animals such as cows and goats raised and used for milk production or for other uses such as sheep sheared for the production of wool. As will be known by those skilled in the art, animal derived platelet lysate (PL) or platelet rich plasma (PRP) from different animals such as bovines, equines and canines have been used for the treatment of a wide range of injuries in veterinary medicine and have been proposed for cultivation of human stem cells as an animal friendly replacement of FBS. In other embodiments, the animals can be raised exclusively for the purpose of sustainably producing blood products.

In some embodiments, the platelet lysate (PL) and/or platelet rich plasma (PRP) can be sustainably harvested from living animals using methods such as apheresis or methods using gravity to separate cells such as centrifugation or sedimentation of red blood cells at atmospheric or high pressure as are known in the art where red and white blood cells are removed from a living animal and plasma and other blood components including platelets, red blood cells, fibrin and other proteins and blood components including platelet and other plasma soluble factors such as fibrin, albumin, minerals, vitamins and growth factors are removed before the remaining blood product is returned to the animal. Such blood derivatives can thereby be obtained from living animals with minimal ethical burden and in a manner similar to that by which sera is regularly obtained from humans. In some embodiments, wasted blood products from slaughterhouses can also be used as an inexpensive secondary source to produce PL and PRP supplement for cultivated meat.

In some embodiments, the platelet lysate (PL) can be produced by treatment of blood platelets isolated from blood and blood sera according to a variety of physical methods. For example, physical treatments such as those selected from the group consisting of freezing and thawing, physical shearing such as by sonication to release their content including cytokines and growth factors, agitation, aging and adhesion of platelets to surfaces, are known to induce platelet lysis or release of growth factor from platelet.

In some embodiments, platelet-rich plasma can be treated using various chemical and biochemical methods known to those of skill in the art in order to releases cytokines and growth factors. Suitable chemical and biochemical treatments include treatment with a member selected from the group consisting of citrate, EDTA, calcium chloride, plasminogen activating factor and thrombin.

In some embodiments, the cell culture growth media can additionally include peptides, vitamins, cytokines and growth factors, synthetic and/or recombinant proteins, and according to one aspect of the invention, one or more of those components can be extracted from animals' blood including as part of the process of obtaining platelet lysate (PL) and platelet rich plasma.

In some embodiments, the cell culture growth media comprises at least one non-human animal blood component and at least one additive. The one non-human animal blood component can be any blood component as described herein, such as a platelet lysate, a platelet-rich plasma, or a plasma product. The at least one non-human animal blood component can be harvested using any technique as described herein, such as by apheresis. In some embodiments, the at least one additive may comprise peptides, vitamins, cytokines and growth factors and may be obtained from blood or synthesized either chemically or biologically, for example, using recombinant technology.

Some aspects of the disclosure are directed toward methods of raising non-human animal cells in culture, for example, in a bioreactor, comprising providing the cell culture growth media which comprises the combination of platelet lysate (PL) with platelet rich plasma (PRP) in order to boost the proliferation and differentiation of the cultured cells. In some embodiments, the cultured cells comprise myoblasts, fibroblasts, adipocyte, vascular, osteoblasts, mammary glands, epithelial cells, tenocyte, keratinocyte, neural cells, embryonic stem cells, mesenchymal stem cells, etc., isolated from vertebrate and invertebrate animals and are raised to produce cultured animal derived products, such as for example, meat (muscles), fat, skin, horn or other organs such as liver and intestine. In some embodiments, the cultured cells comprise mammalian, avian, reptilian, amphibian, avian and fish species. For example, in some embodiments, the culture of non-human animal cells may comprise cow, sheep, goat, swine, deer, camel, whale, fowl, fishes, crabs, shrimps and insects and can be used to produce cultured animal derived products, such as meat products. It should be understood that the cultured animal derived meat products include traditional meat products that are typically produced from cultured muscle, fat and fibroblast cells but can also include organ tissue such as liver and tusks or skin produced by culture of cells, for example, organ cells, epithelium and keratinocytes.

In some embodiments, any cell type known to those of ordinary skill in the art suitable for cell culture can be used, for example, to produce the cell-based meat products. Non-limiting examples, include stem cells, embryonic stem cells, bone marrow derived stem cells, adipose tissue derived stem cells, mesenchymal stem cells and induced pluripotent stem cells.

In some embodiments, the platelet lysate (PL) and/or platelet rich plasma (PRP) in the cell culture growth medium can be extracted from the blood of the same species of the cells being cultivated; in other embodiments, the platelet lysate (PL) and/or platelet rich plasma (PRP) in the cell culture growth medium can be extracted from the blood of one or more different species as the cells being cultivated. According to some embodiments, the platelet lysate and/or platelet rich plasma in the cell culture growth medium can be extracted from the blood of multiple different species and pooled together.

In some embodiments, the disclosure is directed toward a cell culture media for the cultivation of cell-based meat, comprising a platelet lysate and a platelet rich plasma sustainably harvested using non-human animals.

In some embodiments, the disclosure is directed toward a cell culture media for cultivation of cell-based products comprising a platelet lysate and/or platelet rich plasma supplemented by a plurality of exogenous growth factors.

In some embodiments, the disclosure is directed toward a cell culture growth media for cell-based meat production, comprising a platelet lysate, wherein the platelets are harvested from a live animal using apheresis.

In some embodiments, the disclosure is directed toward a cell culture growth media for cell-based meat production, comprising a platelet-rich plasma, wherein the platelet-rich plasma is harvested from a live animal using apheresis.

In some embodiments, the disclosure is directed toward a cell culture growth media for cell-based meat production, comprising a plasma product, wherein the plasma product is harvested from a live animal using apheresis.

In some embodiments, the disclosure is directed toward a cell culture growth media for cell-based meat production comprising at least one non-human animal blood component and at least one additive.

In some embodiments, the disclosure is directed toward a cell culture growth factor supplement comprising platelet lysate (PL) and platelet rich plasma (PRP).

In some embodiments, the disclosure is directed toward an article comprising a cell-based meat product comprising a platelet lysate and a platelet-rich plasma, wherein at least 0.1% of the cells in the cell-based meat product are platelet cells.

In some embodiments, the disclosure is directed toward an article comprising a cell-based meat product comprising a platelet lysate and a plasma product, wherein at least 0.1% of the cells in the cell-based meat product are platelet cells.

In some embodiments, the disclosure is directed toward an article comprising a cell-based meat product comprising a platelet-rich plasma and a plasma product, wherein at least 0.1% of the cells in the cell-based meat product are platelet cells.

In some embodiments, the disclosure is directed toward an article comprising a cell culture medium and a non-human platelet lysate.

In some embodiments, the disclosure is directed toward an article comprising a cell culture medium and a non-human plasma product.

In some embodiments, the disclosure is directed toward an article comprising a cell culture medium and a non-human platelet-rich plasma.

In some embodiments, the disclosure is directed toward an article comprising a cell culture medium, a platelet lysate, and a bovine platelet-rich plasma.

In some embodiments, the disclosure is directed toward an article comprising a cell culture medium, a platelet lysate and a platelet-rich plasma, wherein the platelet lysate and platelet-rich plasma comprise between 2% to 20% by weight of the cell culture medium.

In some embodiments, the disclosure is directed toward an article comprising a cell culture medium, a platelet lysate and a plasma, wherein the platelet lysate and plasma comprise between 2% to 20% by weight of the cell culture medium.

In some embodiments, the disclosure is directed toward an article comprising a cell culture medium, a plasma and a platelet-rich plasma, wherein the plasma and platelet-rich plasma comprise between 2% to 20% by weight of the cell culture medium

In some embodiments, the disclosure is directed toward an article comprising a cell culture medium, a platelet lysate, and a bovine platelet-rich plasma, wherein the bovine platelet-rich plasma comprises 10% by weight of the cell culture medium.

In some embodiments, the disclosure is directed toward an article comprising a cell culture medium, a plasma and a bovine platelet-rich plasma, wherein the bovine platelet-rich plasma comprises 10% by weight of the cell culture medium.

In some embodiments, the disclosure is directed toward a method of raising non-human cells for cell-based products in culture comprising:

exposing a plurality of non-human cells in a bioreactor to a cell culture media comprising a platelet lysate and a platelet rich plasma extracted from non-human animals without slaughtering said non-human animal.

In some embodiments, the disclosure is directed toward a method comprising adding a platelet lysate and a platelet-rich plasma to a bioreactor containing a cell culture growth media, adding animal cells to the bioreactor, and growing a cell-based meat product in the bioreactor.

In some embodiments, the disclosure is directed toward a method comprising adding a plasma to a bioreactor containing a cell culture growth media, adding animal cells to the bioreactor, and growing a cell-based meat product in the bioreactor.

In some embodiments, the disclosure is directed toward a method comprising adding a platelet lysate to a bioreactor containing a cell culture growth media, adding animal cells to the bioreactor, and growing a cell-based meat product in the bioreactor.

In some embodiments, the disclosure is directed toward a method comprising adding a platelet-rich plasma to a bioreactor containing a cell growth media, adding animal cells to the bioreactor, and growing a cell-based meat product in the bioreactor.

In some embodiments, the disclosure is directed toward a method comprising freeze-thawing a donated platelet concentrate to produce a platelet lysate, adding the platelet lysate to a bioreactor containing a cell growth medium, and cultivating a cell-based meat product in the bioreactor.

In some embodiments, the disclosure is directed toward a method comprising harvesting a whole blood sample from a living animal, isolating a platelet rich plasma from the whole blood sample, and adding the platelet-rich plasma to a bioreactor, wherein the bioreactor contains a cell-based meat product.

In some embodiments, the disclosure is directed toward a method comprising harvesting a whole blood sample from a living animal, isolating a plasma product from the whole blood sample, and adding the plasma product to a bioreactor, wherein the bioreactor contains a cell-based meat product.

In another aspect, the present disclosure encompasses methods of making one or more of the embodiments described herein, for example, a cell-culture growth medium. In still another aspect, the present disclosure encompasses methods of using one or more of the embodiments described herein, for example, a cell-culture growth medium.

Other advantages and novel features of the present disclosure will become apparent from the following detailed description of various non-limiting embodiments of the disclosure when considered in conjunction with the accompanying figures.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 depicts the effects of different cell culture growth media on lamb myoblast proliferation, according to some embodiments.

FIGS. 2A and 2B depict the effects of different cell culture growth media on (FIG. 2A) resulting cell counts (FIG. 2B) and doubling time, according to some embodiments;

FIG. 3 illustrates the effect of supplementing platelet-rich plasma with growth factors on the proliferation of bovine myocytes, according to some embodiments;

FIG. 4 illustrates the effect of platelet-rich plasma isolated from different cows on the proliferation of bovine myocytes, according to some embodiments;

FIGS. 5A-E illustrate the effect of varying the concentration of platelet-rich plasma, isolated from various cows, in the culture media on bovine myoblast proliferation. The platelet rich plasma was isolated from (FIG. 5A) cow 2371, (FIG. 5B) cow 4321, (FIG. 5C) cow 4266, (FIG. 5D) cow 4348, and (FIG. 5E) cow 14583, according to some embodiments;

FIGS. 6A-F illustrate the effect of varying the concentration of platelet-rich plasma, isolated from various cows, in the culture media on human hepatocyte proliferation. The platelet rich plasma was isolated from (FIG. 6A) cow 2398, (FIG. 6B) cow 14347, (FIG. 6C) cow 4266, (FIG. 6D) cow 2371, (FIG. 6E) cow 4321, and (FIG. 6F) cow 14424, according to some embodiments;

FIGS. 7A-H illustrate the hematology and blood chemistry results from cow 4266, including: (FIG. 7A) red blood cells (M/microliter), (FIG. 7B) hemoglobin (g/dL), (FIG. 7C) white blood cells (K/microliter), (FIG. 7D) platelets (K/microliter), (FIG. 7E) fibrinogen (mg/dL), (FIG. 7F) albumin (g/dL), (FIG. 7G) aspartate aminotransferase (U/L), and (FIG. 7H) alkaline phosphatase (U/L), according to some embodiments;

FIGS. 8A-H illustrate the hematology and blood chemistry results from cow 4348, including: (FIG. 8A) red blood cells (M/microliter), (FIG. 8B) hemoglobin (g/dL), (FIG. 8C) white blood cells (K/microliter), (FIG. 8D) platelets (K/microliter), (FIG. 8E) fibrinogen (mg/dL), (FIG. 8F) albumin (g/dL), (FIG. 8G) aspartate aminotransferase (U/L), and (FIG. 8H) alkaline phosphatase (U/L), according to some embodiments;

FIGS. 9A-H illustrate the hematology and blood chemistry results from cow 2315, including: (FIG. 9A) red blood cells (M/microliter), (FIG. 9B) hemoglobin (g/dL), (FIG. 9C) white blood cells (K/microliter), (FIG. 9D) platelets (K/microliter), (FIG. 9E) fibrinogen (mg/dL), (FIG. 9F) albumin (g/dL), (FIG. 9G) aspartate aminotransferase (U/L), and (FIG. 9H) alkaline phosphatase (U/L), according to some embodiments;

FIGS. 10A-H illustrate the hematology and blood chemistry results from cow 2371, including: (FIG. 10A) red blood cells (M/microliter), (FIG. 10B) hemoglobin (g/dL), (FIG. 10C) white blood cells (K/microliter), (FIG. 10D) platelets (K/microliter), (FIG. 10E) fibrinogen (mg/dL), (FIG. 10F) albumin (g/dL), (FIG. 10G) aspartate aminotransferase (U/L), and (FIG. 10H) alkaline phosphatase (U/L), according to some embodiments;

FIGS. 11A-H illustrate the hematology and blood chemistry results from cow 5211, including: (FIG. 11A) red blood cells (M/microliter), (FIG. 11B) hemoglobin (g/dL), (FIG. 11C) white blood cells (K/microliter), (FIG. 11D) platelets (K/microliter), (FIG. 11E) fibrinogen (mg/dL), (FIG. 11F) albumin (g/dL), (FIG. 11G) aspartate aminotransferase (U/L), and (FIG. 11H) alkaline phosphatase (U/L), according to some embodiments;

FIGS. 12A-H illustrate the hematology and blood chemistry results from cow 5276, including: (FIG. 12A) red blood cells (M/microliter), (FIG. 12B) hemoglobin (g/dL), (FIG. 12C) white blood cells (K/microliter), (FIG. 12D) platelets (K/microliter), (FIG. 12E) fibrinogen (mg/dL), (FIG. 12F) albumin (g/dL), (FIG. 12G) aspartate aminotransferase (U/L), and (FIG. 12H) alkaline phosphatase (U/L), according to some embodiments;

FIGS. 13A-H illustrate the hematology and blood chemistry results from cow 14424, including: (FIG. 13A) red blood cells (M/microliter), (FIG. 13B) hemoglobin (g/dL), (FIG. 13C) white blood cells (K/microliter), (FIG. 13D) platelets (K/microliter), (FIG. 13E) fibrinogen (mg/dL), (FIG. 13F) albumin (g/dL), (FIG. 13G) aspartate aminotransferase (U/L), and (FIG. 13H) alkaline phosphatase (U/L), according to some embodiments;

FIGS. 14A-H illustrate the hematology and blood chemistry results from cow 14583, including: (FIG. 14A) red blood cells (M/microliter), (FIG. 14B) hemoglobin (g/dL), (FIG. 14C) white blood cells (K/microliter), (FIG. 14D) platelets (K/microliter), (FIG. 14E) fibrinogen (mg/dL), (FIG. 14F) albumin (g/dL), (FIG. 14G) aspartate aminotransferase (U/L), and (FIG. 14H) alkaline phosphatase (U/L), according to some embodiments;

FIGS. 15A-15L illustrate experiments involving multiple applications of apheresis on cows bi-weekly or weekly, showing that such collections did not substantially affect certain biomarkers related to the health of the cows, in another embodiment;

FIG. 16 shows the concentration of fibroblast growth factor-2 in the blood following weekly or biweekly blood draws from a young Holstein Heifer, a young Steer, and a mature Holstein, in some embodiments;

FIG. 17 shows the concentration of insulin growth factor in the blood following weekly or biweekly blood draws from a young Holstein Heifer, a young Steer, and a mature Holstein, in some embodiments;

FIG. 18 illustrates the cell proliferative effects of adding 10% platelet-rich plasma, isolated from various cows to a culture of bovine myoblasts. The platelet rich plasma was isolated weekly from Cow 4266, Cow 4321, and 4348. The platelet rich plasma was isolated biweekly from Cow 14347, Cow 14424, and Cow 14583, according to some embodiments;

FIG. 19 compares the cell proliferative effects of adding 10% platelet-rich plasma to a culture of bovine myoblasts. Platelet-rich plasma was isolated from either mature (2371, 2348, and 2315) or young (14347, 14424, and 14583) cows; according to some embodiments; and

FIG. 20 shows the effect of platelet-rich plasma isolated from either male or female cows on the proliferation of bovine myoblasts; according to some embodiments.

### DETAILED DESCRIPTION

Cell-based cultivated meat is a cultured animal derived product and is a recent innovation in the food industry. Cell-based meat is manufactured using animal cells (typically non-human) under in vitro conditions and using a cell culture medium to create a meat without farming animals in the traditional way. The term cell-based meat is synonymous with cultivated meat, cultured meat, cellular meat, slaughter-free meat, and synthetic meat, among other related terms.

Thus, the present disclosure generally relates to an improved cell-culture growth medium, for example, for the production of cell-based meat, and other cultivated products, or applications. For example, certain embodiments are directed to a cell culture growth factor supplement comprising platelet lysate (PL) and platelet rich plasma (PRP). Such solutions may be used, for example, to increase the cellular biomass in a bioreactor by enhancing the rate and frequency of cellular proliferation. In some embodiments, platelet-rich plasma is isolated from the whole blood of a living animal. The platelets within the platelet-rich plasma may be concentrated, for example, by centrifugation, to produce a platelet concentrate. In some embodiments, the platelet concentrate can be cultivated and activated using an agonist to release growth factors. In some embodiments, the growth factor solution can be separated from the platelets and added to a bioreactor containing a cell-based meat product. In some embodiments, the platelet concentrate can be lysed to produce a platelet lysate. In some embodiment a cell-culture media comprising a platelet lysate and platelet-rich plasma can be added to a bioreactor. Other embodiments are generally directed towards compositions and methods of use of the platelet lysate and/or platelet-rich plasma, cultivated meat products using these, kits involving these, or the like.

Cell-based meats are comprised of cellular biomass. Technologies that maximize production of cellular biomass in a sustainable and cost-effective manner are thus desirable. The current gold standard in the field is to use fetal bovine serum to stimulate cell proliferation of non-human animal cells to produce the cellular biomass. Fetal bovine serum contains growth factors that stimulate a number of different mammalian cell types, e.g., myoblasts, stem cells, etc. The use of fetal bovine serum, however, is controversial, as it is obtained from a bovine fetus, via a closed collection system, at a slaughterhouse. Further, harvesting fetal bovine serum requires large-scale animal husbandry facilities that have been linked to public health complications, environmental degradation, and animal welfare concerns.

As such, some aspects of the present disclosure are directed toward procuring growth factors in a safe, humane, and environmentally sustainable manner. For example, in some embodiments the growth factors can be obtained, either directly or indirectly, from a whole blood sample taken from a living animal, such as a cow, pig, goat, etc., not intended for slaughter. In some embodiments, the whole blood sample can be separated, for example, by apheresis or centrifugation, into blood components, e.g., blood plasma, platelet-rich plasma, platelet concentrate, etc. As will be appreciated by those skilled in the art, blood plasma and/or platelet-rich plasma, which contain the necessary growth factors to stimulate cell proliferation, can be added directly to a basal cell culture media to produce a cell-culture growth media, for example, for cultivation of a cell-based meat product.

In other embodiments, the platelet-rich plasma can be activated to release at least one growth factor. The platelets may be activated in some cases, for example, by exposure to an antigen or an agonist. In some embodiments, activating the platelet-rich plasma produces a serum solution comprising at least one growth factor. Such solutions can be, for example, added to a cell culture media and used to stimulate cell proliferation of myoblasts, and other non-human animal cells, for production of cell-based meats. In some embodiments, the serum solution can be separated from the activated platelets and added to a cell culture solution to produce a cell-culture growth media.

Growth factors can also be produced, for example, by lysing the platelets within the platelet-rich plasma or from a donated platelet concentrate, to produce a platelet lysate. Platelets can be lysed using any technique known to those skilled in the art, for example, freeze-thawing, osmotic imbalance, acoustic cavitation, extrusion, etc. For example, the cells may be repeatedly extruded through a porous membrane, which exerts high shear forces on the cells, thereby causing the cell to burst, thus releasing the growth factors.

Other aspects of the disclosure are directed toward repeated blood collection from non-human animals, for example, to obtain platelet rich plasma or a platelet lysate, etc. For example, in some embodiments, blood may be withdrawn from the animal at spaced intervals, so as to allow the animal time to recover and produce new blood. For instance, blood may be withdrawn from the animal every 2 weeks, every 4 weeks, every 6 weeks, every 2 months, or the like. The blood draws may be processed, for example, as discussed herein. For example, the blood may be used to obtain platelet rich plasma to stimulate cell growth in a bioreactor, e.g., as discussed herein. In this way, such cells can be obtained in certain embodiments in a sustainable and cost-effective manner, e.g., without killing the animal. This usage may result, in certain embodiments, in the reduction in carbon emissions, water use, land use, etc.

The above discussion is a non-limiting example of one embodiment of the present disclosure generally directed to a cell-culture growth medium, for example, based on platelet rich plasma, which can be used for increasing cellular biomass in a bioreactor. However, other embodiments are also possible. Accordingly, more generally, various aspects of the present disclosure are directed to various cell culture growth mediums, for example, for use during the cultivation of meat and other cultivated products.

Thus, for example, some aspects of the present disclosure are directed to enhancing cell proliferation using a platelet-rich plasma and/or platelet lysate, for example, in bioreactors containing products such as cultivated meat products, or other bioreactors or applications. For instance, some embodiments are directed toward activating cultures of platelet rich plasma and/or platelet concentrates to produce a serum solution comprising at least one growth factor. In some cases, the cells can be filtered out of the serum solution. This serum solution (with or without cells) can be used in a variety of applications. For instance, it may be used within the same or a different bioreactor to enhance cell adhesion and proliferation. Other examples of suitable applications include, but are not limited to, biopharmaceuticals, animal furs, cell-based organs, etc., which can be manufactured, for example, as discussed herein.

The serum solution may be prepared and used as discussed herein, e.g., relatively soon after preparation. However, in some embodiments, the serum solution can be stored for at least 1 week, at least 2 weeks, at least 1 month, at least 6 months, at least 12 months, etc. at room temperature or other temperatures, e.g., at 4° C., at 0° C., at −4° C., at −20° C., etc. As a non-limiting example, the serum solution may be freeze-dried in some embodiments. In addition, in some embodiments, the freeze-dried serum solution can be reconstituted, e.g., at its original concentration, or at higher or lower concentrations, such as at concentrations that are at least 10×, at least 50×, or at least 100× as concentrated as the original serum solution.

Certain aspects of the disclosure are directed toward obtaining growth factors and/or other components from various blood components, e.g., platelets, plasma, and/or platelet-rich plasma can be isolated in some embodiments directly from the blood of a non-human animal, e.g., cow, pig, sheep, goat, deer, fish, duck, turkey, shrimp, etc.

Thus, platelet rich plasma (PRP) can be derived from whole blood from which red blood cells have been removed, such as by centrifugation. Plasma can also be derived from whole blood by, for example, using apheresis or by removing platelets from the platelet-rich plasma product using, for example, centrifugation. Plasma and platelet rich plasma (PRP) contain a variety of growth factors that are in blood such as transforming growth factor beta, fibroblast growth factor, insulin-like growth factor 1, insulin-like growth factor 2, vascular endothelial growth factor, epidermal growth factor, interleukin 8, keratinocyte growth factor and connective tissue growth factor. In addition, lysing the platelets in the platelet-rich plasma can release platelet-derived growth factor. Platelet rich plasma (PRP) can be categorized based on its leukocyte and fibrin content as leukocyte-rich PRP (L-PRP), leukocyte reduced PRP (P-PRP); leukocyte reduced or pure PRP, (4) leukocyte platelet-rich fibrin and pure platelet-rich fibrin (L-PRF). As used herein, “platelet-rich plasma” (“PRP”) is plasma having platelets at a concentration of at least 2×, at least 5×, or at least 10× the normal concentration of platelets in blood. A “platelet-poor plasma” is a plasma comprising some platelets, but at a concentration that is less than the normal concentration of platelets in blood.

Plasma and/or platelet-rich plasma contain growth factors, and other solutes, known by those skilled in the art, to enhance biomass production. For example, plasma and/or platelet rich plasma comprises adhesive proteins, e.g., fibrinogen, which can facilitate non-human cells, e.g., myoblasts, to adhere to microcarriers, e.g., fibrin microcarriers; it also comprises growth factors, e.g., platelet-derived growth factor that enhance cell proliferation. Other solutes of plasma and/or platelet-rich plasma include dissolved proteins (6-8% by weight), e.g., serum albumins, goblins, and fibrinogen), glucose, clotting factors, electrolytes (Na⁺, Ca²⁺, Mg²⁺, HCO₃⁻, Cl⁻, etc.), and hormones, etc. As such, in some embodiments, the plasma and/or platelet-rich plasma can be added to any cell culture media to produce a cell culture growth medium and to grow non-human cells, for example, in a bioreactor to produce a cell-based meat product. For example, in some cases plasma and/or platelet-rich plasma can be added to any cell culture media, such as DMEM or Essential 8, to produce a cell culture growth medium, and added to a bioreactor, for example, containing a cell-based meat product.

In some instances, it may be desirable to obtain a platelet concentrate directly from whole blood, for example, by apheresis or from a platelet-rich plasma by, for example, centrifugation, or other techniques known to those of skill in the art, e.g., tangential flow filtration. In some embodiments, the final platelet concentration in platelet-rich plasma is at least 10⁵ platelets/mL, at least 10⁶ platelets/mL, at least 10⁷ platelets/mL, at least 10⁸ platelets/mL, at least 10⁹ platelets/mL, at least 10¹⁰ platelets/mL, etc., in the platelet concentrate.

In some cases, it may be desirable to activate the platelets within the platelet-rich plasma and/or the platelet concentrate to produce a serum solution comprising at least one growth factor (e.g., to enhance the growth of animal cells in a bioreactor). For example, in some embodiments, an agonist can be used to activate the platelets. Any agonist known by those skilled in the art can be used to produce the activated platelets. Non-limiting examples of agonists that can be used to produce activated platelets include adenosine diphosphate (ADP), thromboxane, thrombin, epinephrine, phorbol 12-myristate 13-acetate, thrombin-receptor agonist peptide, and the like. In some embodiments, the platelet-rich plasma and/or platelet concentrate can be cultured prior to activation; in other embodiments, the platelet-rich plasma and/or platelet concentrate can be cultured after activation. In some embodiments, a suspension of platelet-rich plasma and/or platelet concentrate can be activated without culturing.

In some embodiments, the concentration of agonist needed to activate the platelet-rich plasma and/or the platelet concentrate is at least 1 microgram/mL, at least 5 microgram/mL, at least 10 microgram/mL, at least 40 microgram/mL, at least 80 microgram/mL, at least 100 microgram/mL, at least 500 microgram/mL, at least 1 mg/mL, at least 10 mg/mL, at least 50 mg/mL, at least 100 mg/mL, at least 500 mg/mL, at least 1 g/mL, etc. In addition, in some embodiments, the concentration of agonist may be no more than 1 g/mL, no more than 500 mg/mL, no more than 100 mg/mL, no more than 50 mg/mL, no more than 10 mg/mL, no more than 1 mg/mL, no more than 500 microgram/mL, no more than 400 microgram/mL, no more than 300 microgram/mL, no more than 100 microgram/mL, no more than 80 microgram/mL, no more than 40 microgram/mL, no more than 10 microgram/mL, no more than 5 microgram/mL, no more than 1 microgram/mL, etc. In addition, combinations of any of these ranges are also possible in certain embodiments. If more than one agonist is present, they may independently have the same or different concentrations.

In certain embodiments, antigens can be used to activate the platelet-rich plasma and/or platelet concentrate. Exemplary antigens include exotoxins, such as botulinum toxin produced by Clostridium botulinum, and endotoxins (e.g., lipopolysaccharide complexes, (LPS)), for example, that are associated with the outer membrane of Gram-negative pathogens such as Escherichia coli, Salmonella, Shigella, pseudomonas, and the like.

In some embodiments, the concentration of antigen needed to activate the platelet-rich plasma and/or the platelet concentrate is at least 1 microgram/mL, at least 5 microgram/mL, at least 10 microgram/mL, at least 40 microgram/mL, at least 80 microgram/mL, at least 100 microgram/mL, at least 500 microgram/mL, at least 1 mg/mL, at least 10 mg/mL, at least 50 mg/mL, at least 100 mg/mL, at least 500 mg/mL, at least 1 g/mL, etc. In addition, in some embodiments, the concentration of antigen may be no more than 1 g/mL, no more than 500 mg/mL, no more than 100 mg/mL, no more than 50 mg/mL, no more than 10 mg/mL, no more than 1 mg/mL, no more than 500 microgram/mL, no more than 400 microgram/mL, no more than 300 microgram/mL, no more than 100 microgram/mL, no more than 80 microgram/mL, no more than 40 microgram/mL, no more than 10 microgram/mL, no more than 5 microgram/mL, no more than 1 microgram/mL, etc. In addition, combinations of any of these ranges are also possible in certain embodiments. If more than one antigen is present, they may independently have the same or different concentrations.

In some embodiments, the platelet-rich plasma and/or platelet concentrate can be activated by exposure to a sheer force. For example, bioreactors that use propellers for mixing exert shear forces on the cells within the bioreactor. In some embodiments, the cells may be activated by exposing the cells to shear forces of at least 5 dynes/cm squared, at least 10 dynes/cm squared, at least 15 dynes/cm squared, at least 20 dynes/cm squared, at least 25 dynes/cm squared, at least 30 dynes/cm squared, at least 35 dynes/cm squared, at least 40 dynes/cm squared, at least 45 dynes/cm squared, at least 50 dynes/cm squared, etc.

In some embodiments, activated platelet-rich plasma and/or platelet concentrate can be produced by allowing such cells to adhere to a substrate, e.g., a fibrin microcarrier in the bioreactor. Non-limiting examples of microcarriers include those described in U.S. Pat. Apl. Ser. No. 63/159,403, incorporated herein by reference in its entirety.

In some embodiments, a microcarrier, e.g., a fibrin microcarrier, can be added to the bioreactor to activate the platelets. In some embodiments, the concentration of microcarriers needed to activate the platelets is at least 10 mg/mL, at least 40 mg/mL, at least 80 mg/mL, at least 100 mg/mL, at least 500 mg/mL, at least 1 g/mL, at least 5 g/mL, at least 10 g/mL, etc. In addition, in some cases, the concentration may be no more than 10 g/mL, no more than 5 g/mL, no more than 1 g/mL, no more than 500 mg/mL, no more than 100 mg/mL, no more than 80 mg/mL, no more than 40 mg/mL, no more than 10 mg/mL, etc. In addition, combinations of any of these ranges are also possible in certain cases.

In addition, it should be understood that in certain embodiments, the platelet-rich plasma and/or platelet concentrate may be activated by using any combination of the activation techniques described herein. For example, the platelets may be activated using any combination of agonists, antigens, sheer force, adhesion to a substrate, etc.

It should also be understood, however, that not all the platelets will become activated following treatment with one, or more, of the methods described herein. For example, in some embodiments, activated platelets may contain at least 1%, at least 5%, at least 10%, at least 20%, at least 30%, at least 40%, at least 50% and/or no more than 50%, no more than 40%, no more than 30%, no more than 20%, no more than 10%, no more than 5%, no more than 1%, etc. of non-activated platelets. In other cases, however, substantially all of the blood platelets may be activated.

In some instances, it may be desirable to lyse the platelets to produce a platelet lysate. In some embodiments, the platelet lysate comprises a non-human platelet-lysate; in other embodiments, the platelet lysate comprises a human platelet lysate. As described elsewhere herein, platelets contain granules, which store growth factors, and other solutes, known by those skilled in the art, to promote cell adhesion and proliferation. Therefore, some embodiments are directed toward producing a platelet lysate comprising at least one growth factor. In some embodiments, the platelet rich plasma and/or platelet concentrate may be lysed, for example, and added to a cell culture medium.

Accordingly, it should be understood that, in some embodiments, the platelet lysate may be obtained from a donated platelet concentrate, such as a donated human platelet concentrate, obtained, for example, from a medical facility, such as a hospital. In other embodiments, the platelet concentrate may be obtained, for example, from a veterinary hospital or slaughterhouse. In some embodiments, the concentration of platelets in the platelet concentrate is at least 10⁹ platelets per mL and comprises at least 5×10¹⁰ platelets. However, it will be appreciated that these values are merely non-limiting examples. For example, in some cases, the concentration of platelets may be at least 10⁵, at least 10⁶, at least 10⁷, at least 10⁸, at least 10⁹, at least 10¹⁰ platelets/mL, etc. In some cases, there may be at least 10⁵, at least 10⁶, at least 10⁷, at least 10⁸, at least 10⁹, at least 10¹⁰ platelets, etc., within a given sample.

Those of ordinary skill in the art will appreciate that donated platelet concentrates may have a useful life span of about 7 days after donation and after expiration of this period the platelet concentrate can be frozen and thawed, which may lyse some or all of the cells. In some cases, the lifespan may be at least 3 days, at least 4 days, at least 5 days, at least 6 days, at least 7 days, at least 8 days, at least 9 days, at least 10 days, etc.

In some embodiments, the platelet concentrate can be obtained using a two-step process in which multiple centrifugations are carried out on a whole blood sample. In the first centrifugation, the platelet rich plasma (PRP) is separated from the whole blood. In some embodiments, the concentration of the platelets is about 3×10⁸ platelets per mL after the first centrifugation. In some embodiments, the platelet concentration in the platelet rich plasma fraction can be increased by a second centrifugation step. In some embodiments, the concentration of platelets after the second centrifugation step is at least 10⁹ or more platelets. However, it will be understood that this is just one non-limiting example, and other techniques of obtaining platelets from a blood sample can be used in other embodiments, for example, apheresis or other techniques such as those described herein. In addition, the concentration of platelets obtained after centrifugation may vary; for example, the concentration may be any of the platelet concentrations described above.

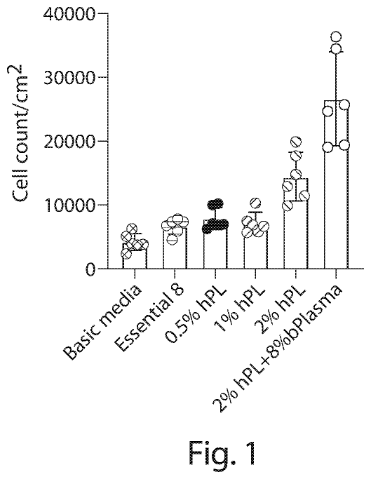

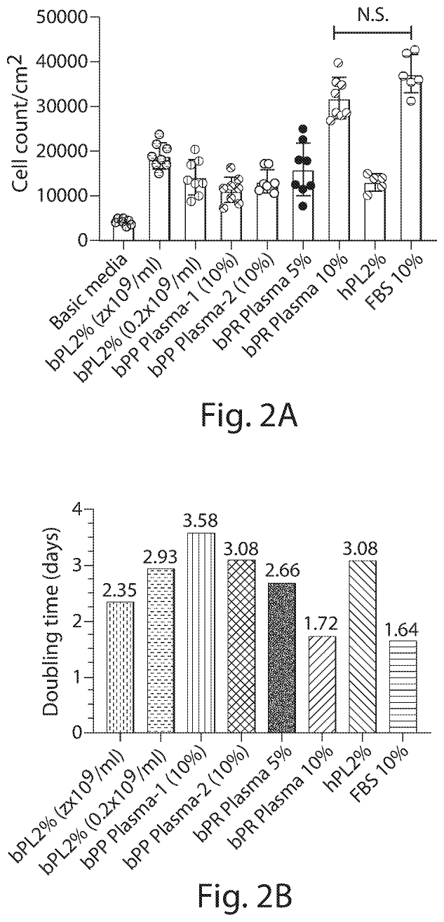

It has been found that, in accordance with one set of embodiments, PRP production from bovine blood is easier, more economical and yields improved cell proliferation results compared to concentrated platelet lysate. Thus, in some embodiments, the platelet rich plasma comprises bovine platelet rich plasma. For example, the data in FIGS. 2A and 2B show that cell culture growth media comprising bovine PL at a 2% concentration provides similar cell growth efficacy regardless of the initial platelet counts, but addition of bovine PRP at concentrations up to 10% of the cell culture growth media increases the proliferation rate. The data presented in FIG. 1 also indicates that the addition of bovine plasma to a human platelet lysate, improves the efficacy of PL up to two times. These data highlight that the presence of growth factors, cytokines, proteins, vitamins and minerals in plasma are of significant importance to the proliferation of cells. Accordingly, in some embodiments, the cell culture growth media comprises bovine platelet rich plasma and human platelet lysate. However, these data are intended to be exemplary and not limiting.

As mentioned, certain embodiments are generally directed to producing lysate, e.g., a platelet lysate. For example, in one set of embodiments, cells may be lysed by exposing them to hypoosmotic water, such as distilled water. Accordingly, in certain embodiments, the platelet-rich plasma and/or platelet concentrate is exposed to a hypoosmotic solution to cause the cells to lyse. In some embodiments the cells may be exposed to a volume of hypoosmotic water that is at least sufficient to lyse the platelets. For example, the volume may be at least 5%, at least 10%, at least 20%, at least 30%, at least 40%, or at least 50% of the volume of the solution containing the cells. These percents are percent by volume.

In addition, other methods of lysing cells can be used in other embodiments. As one non-limiting example, acoustic energy may be used to lyse the cells. For example, the cells may be exposed to a sonicator or an ultrasonic bath to cause the cells to lyse. In another set of embodiments, the cells may be exposed to one or more freeze-thaw cycles, e.g., where the temperature is lowered to below the freezing point of the solution, thereby causing ice to form, killing at least some of the cells. As a third example, in some embodiments, the platelet lysate may be produced by agitating the blood platelets. As a fourth example, the platelet lysate may be produced by aging the blood platelets for at least 5 days. As a fifth example, the platelet lysate may be produced by homogenizing blood platelets, in accordance with another set of embodiments. In yet another embodiment, cells may be passed through an extrusion membrane, e.g., repeatedly, where the shear stress induced during passage through the membrane pores lyses the cells.

In certain embodiments, the cells may be lysed such that at least 50% of the cells have been lysed or ruptured, and in some embodiments, such that at least 60%, at least 70%, at least 80%, at least 85%, at least 90%, at least 95%, or at least 99% of the cells have been lysed or ruptured. Further, one or more lysing technique may be used in various embodiments

In some instances, it may be desirable to separate the platelet-rich plasma and/or platelet concentrate from the platelet lysate or the serum solution comprising at least one growth factor. In some embodiments, the platelets may be separated from the lysate or serum solution comprising at least one growth factor using any technique known to those skilled in the art. Such techniques may include, for example, sedimentation, filtration, sorting, centrifugation, immune-density cell separation, chromatography, etc. In some cases, the platelets may be separated using filtration techniques, such as gravity filtration, vacuum filtration, cold filtration, hot filtration, multi-layer filtration, mechanical filtration, surface filtration, depth filtration, tangential flow filtration, and the like. In some embodiments, the platelets may be separated using cell-sorting techniques such as fluorescence-active cell sorting (FACS), microfluidic cell sorting, or the like. In other embodiments, the cells can be separated using, for example, density gradient centrifugation or affinity chromatography. In some embodiments, the serum solution can be separated from the platelets by decanting.

In some cases, the platelet lysate or the serum solution may be stored at room or ambient temperatures, in a refrigerator or a freezer, etc. In some embodiments, the plasma and/or serum solution can be stored for at least 1 days, for at least 2 days, for at least 3 days, for at least 4 days, for at least 5 days, for at least 6 days, for at least 7 days, for at least 10 days, for at least 14 days, etc., for example, when stored at such temperatures.

In some embodiments, the platelet lysate, plasma, or the serum solution comprising the growth factors can be freeze-dried. Those of ordinary skill in the art will know of systems and methods for freeze drying compounds. In addition, in some embodiments, the freeze-dried solution can be reconstituted, e.g., at its original concentration, or at higher or lower concentrations, such as at concentrations that are at least 10×, at least 50×, at least 100×, at least 200×, at least 300×, at least 400×, at least 500×, at least 600×, at least 700×, at least 800×, at least 900×, at least 1000× as concentrated as the original solution.

In addition, certain aspects of the disclosure are directed toward producing cell-culture growth media, for example, for cultivation of cell-based meats, or other animal-derived products, or other techniques such as those described herein. In some embodiments, the cell-culture growth media comprises a cell-culture media. Exemplary cell-culture medias that may be purchased by commercial vendors (e.g., Gibco, Sartorius, etc.), or synthetized by those skilled in the art, include DMEM, RPMI 1640, MEM, DMEM/F12, Ham's F-10 nutrient mixture, Ham's F-12 nutrient mixture, Media 199, Plasma-Lyte, PBS, etc.

In some instances, it may be desirable to add one or more blood components, e.g., plasma, serum, platelet-rich plasma, platelet lysate, etc., to the cell-culture media, for example, to enhance the biological activity of the cell-culture media, e.g., to promote cell proliferation. In some embodiments, the blood components, e.g., plasma, serum, platelets, platelet-rich plasma, etc., may arise from non-human living animals selected from the group consisting of cow, sheep, goat, swine, deer, camel, whale, fowl, fish, crab, and shrimp. In some embodiments, the cell culture media comprises a platelet lysate, wherein the platelets are harvested from the live animal using apheresis; in another set of embodiments, the cell culture media comprises a platelet-rich plasma, wherein the platelet-rich plasma is harvested from a live animal using apheresis. In other embodiments, the cell culture growth media comprises a plasma product, wherein the plasma product is harvested from a live animal using apheresis.

In some embodiments, the concentration of platelet-rich plasma and/or platelet lysate is at least 2% by weight, at least 5% by weight, at least 10% by weight, at least 15% by weight, at least 20% by weight, etc. of the cell-culture growth medium. In some embodiments, the concentration of platelet lysate and platelet-rich plasma is at least 2% to 20% by weight of the cell culture media. In other embodiments, the concentration of platelet lysate and platelet-rich plasma is at least 5% to 15% by weight of the cell culture media. In some embodiments, the platelet lysate and the platelet-rich plasma is at least 10% by weight of the cell culture media. In some embodiments, the concentration of plasma and/or serum is at least 2% by weight, at least 5% by weight, at least 10% by weight, at least 15% by weight, at least 20% by weight, etc. of the cell-culture growth medium.

In some embodiments, a cell culture growth medium may comprise a human platelet lysate; in other embodiments, the cell culture growth medium may comprise a bovine platelet-rich plasma. In some cases, according to some embodiments, the cell culture medium may comprise the human platelet lysate and the bovine platelet-rich plasma. In some embodiments, the concentration of human platelet lysate in the cell culture growth medium is at least 1% by weight, at least 2% by weight, at least 3% by weight, at least 5% by weight, at least 10% by weight, etc. In some embodiments, the concentration of bovine platelet rich plasma in the cell culture growth medium is at least 2% by weight, at least 5% by weight, at least 10% by weight, at least 15% by weight, and at least 20% by weight in the cell-culture growth medium. In some embodiments, the ratio of human platelet lysate to bovine platelet-rich plasma in the cell culture growth medium is at least 1:2, at least 1:4, at least 1:6, at least 1:8, at least 1:10, or at least 1:12, etc.

In some embodiments, the cell culture media may comprise platelets. As described elsewhere herein, platelets may be harvested from a non-human living animal such as a cow, a sheep, a goat, a pig (i.e., swine), a deer, a camel, a whale, a fowl, a fish, a crab, or a shrimp. However, in some embodiments, the platelets may be donated by a medical facility (e.g., human platelets), veterinary facility, or a slaughterhouse. In some embodiments, the platelets are harvested from non-human living animals, such as a cow, using apheresis, wherein a small percentage of platelets are removed from the animal with the remaining blood components (e.g., red blood cells, plasma, etc.,) being returned to the animal.

As described elsewhere herein, an agonist and/or antigen may be added to the cell culture media to active the platelets. Non-limiting examples of agonists that may be added to the cell culture media to active the platelets include adenosine diphosphate (ADP), thromboxane, thrombin, epinephrine, phorbol 12-myristate 13-acetate, thrombin-receptor agonist peptide, and the like.

In some embodiments, the cell culture media may comprise a plurality of additives. The additives may be naturally occurring (i.e., found in nature) or synthetic (i.e., man-made). For example, in some embodiments, the cell culture growth media may comprise peptides, vitamins, cytokines and growth factors; in other embodiments, the cell culture media may comprise synthetic and/or recombinant proteins, vitamins, cytokines, and growth factors. Non-limiting examples of other additives include citrate, EDTA, calcium chloride, basic fibroblast growth factor, and plasminogen. In addition, combinations of the additives are also possible in certain embodiments. If more than one additive is present, they may independently have the same or different concentrations. In some embodiments, the additive is selected from the group consisting of proteins, peptides, vitamins, cytokines, and growth factors. In some embodiments, the additive is a synthesized compound; in other embodiments it's a recombinant compound. In some embodiments, the growth factor. Non-limiting examples of growth factors, for example, include transforming growth factor beta, fibroblast growth factor, insulin-like growth factor 1, insulin-like growth factor 2, vascular endothelial growth factor, epidermal growth factor, interleukin 8, keratinocyte growth factor and connective tissue growth factor. In some embodiments, the concentration of growth factor in the cell culture growth media is at least 1 ng/mL, at least 2 ng/ml, at least 5 ng/mL, at least 100 ng/ml, at least 1000 ng/mL. In some embodiments, the concentration of fibroblast growth factor in the cell culture growth media is no greater than 1000 ng/ml, no greater than 100 ng/mL, no greater than 5 ng/mL, no greater than 2 ng/mL, and no greater than 1 ng/mL. In some embodiments, the growth factor is fibroblast growth factor. Additionally, the cell culture growth media may further comprise at least one non-human animal blood component, as described herein. In some embodiments, the at least one non-human animal blood component comprises a platelet-rich plasma; in other embodiments it comprises a plasma product; and its other embodiments still it comprises a platelet lysate.

In some cases, it may be desirable to produce a cell culture growth medium that does not contain platelets (although in other cases, the cell culture growth medium may contain platelets). Therefore, in some embodiments, plasma and/or a serum solution (or a concentrated form of the serum solution), comprising at least one growth factor, can be added to a cell culture medium to produce a cell culture growth medium. In some embodiments, the concentration of the plasma and/or serum solution (or a concentrated form of the serum solution) is at least 2% by weight, at least 5% by weight, at least 10% by weight, at least 15% by weight, or at least 20% by weight of the cell-culture growth medium.

Significantly, it has been found that, in one set of embodiments, increasing the concertation of PRP in a cell culture beyond a certain level does not necessarily yield better results. Thus, in some embodiments, increasing PRP concentrations from 10% to 20% and 30% does not increase the proliferation of animal cells, such as for example, lamb myofibroblast cells. However, in other embodiments, increasing the concertation of PRP in a cell culture beyond a certain level may yield better results.

In one set of embodiments, the cell culture growth medium comprises a cell culture medium and a platelet lysate; in a subset of embodiments, the cell culture growth medium further comprises plasma and/or a platelet-rich plasma. In second set of embodiments, the cell culture medium comprises a cell culture medium and a plasma; in a subset of embodiments, the cell culture medium further comprises a platelet lysate and/or a platelet-rich plasma. In a third set of embodiments, the cell culture medium comprises a cell culture medium and a platelet-rich plasma; in a subset of embodiments, the cell culture growth medium further comprises the plasma and/or the platelet lysate. In another set of embodiments, the cell culture growth medium comprises a cell culture medium, a platelet lysate, and a bovine platelet-rich plasma; in a subset of embodiments, the platelet lysate is a non-human platelet lysate.

Other compositions of the cell culture growth media are also possible. For example, in some embodiments, the cell culture growth medium may comprise a cell culture medium, a platelet lysate, and a platelet-rich plasma, wherein the platelet lysate and platelet-rich plasma comprise between 2% to 20% by weight of the cell culture medium. In other embodiments, the cell culture growth medium comprises the cell culture medium, the platelet lysate and plasma, wherein the platelet lysate and plasma comprise between 2% to 20% by weight of the cell culture medium. In another set of embodiments, the cell culture growth medium comprises plasma and a platelet-rich plasma, wherein the plasma and platelet-rich plasma comprise between 2% to 20% by weight of the cell culture medium. In another non-limiting embodiment, the cell culture growth media may comprise a plasma and a bovine platelet-rich plasma, wherein the bovine platelet-rich plasma comprises 10% by weight of the cell culture medium.

Other aspects of the disclosure are directed toward methods of culturing cells in a cell culture growth media comprising at least one blood component, for example, to produce a cell-based meat product. In some embodiments, the method comprises exposing a plurality of non-human cells in a bioreactor to a cell culture media comprising a platelet lysate and platelet rich plasma extracted from non-human animals without slaughtering said non-human animal. In some embodiments, the cell culture media further comprises a plasma product, such as a bovine plasma product. In addition, combinations are also possible in certain embodiments; for example, in some embodiments the cell culture media comprises a plasma. In other embodiments, the cell culture media comprises a platelet rich plasma and/or a platelet lysate.

Any type of non-human cell known to those of skill in the art can be cultured using the cell culture growth medium comprising the platelet lysate and platelet-rich plasma, including for example, myoblasts, fibroblasts, adipocytes, vascular cells, mammary glands, epithelial cells, osteoblasts, tenocyte neural cells, or the like. In some embodiments, the non-human cells are selected from the group consisting of stem cells, mesenchymal stem cells, and induced pluripotent stem cells. Further, the non-human cells can arise from any animal including, for example, a cow, a sheep, a goat, a swine, a deer, a camel, a whale, a fowl, a fish, a crab, a shrimp or an insect.

In some embodiments, the platelet lysate and platelet-rich plasma in the cell growth media arise from the same animal species as the non-human cells; in other embodiments, the platelet lysate and platelet rich plasma in the cell growth media arise from a different species as the non-human cells. In another set of embodiments, the platelet lysate and platelet rich plasma in the cell growth media arise from multiple different species.

Other methods for cultivating cells are also possible, in other embodiments. For example, in some embodiments, the method comprises adding a platelet lysate and a platelet-rich plasma to a bioreactor containing a cell culture growth media, adding animal cells to the bioreactor, and growing a cell-based meat in the bioreactor. Another method, according to some embodiments, comprises adding plasma to the bioreactor containing a cell culture growth media, adding animal cells to the bioreactor, and growing a cell-based meat product in the bioreactor. A third method, comprises adding a platelet lysate to a bioreactor containing a cell culture growth media, adding animal cells to the bioreactor, and growing a cell-based meat product in the bioreactor, in accordance with other embodiments. A fourth method, according to another set of embodiments, comprises adding the platelet-rich plasma to a bioreactor containing a cell growth media, adding the animal cells to the bioreactor, and growing a cell-based meat product in the bioreactor. A fifth method for cultivating cells, according to some embodiments, comprises freeze-thawing a donated platelet concentrate to produce a platelet lysate, adding the platelet lysate to a bioreactor containing a cell growth media, and cultivating a cell-based meat product in the bioreactor. In other embodiments, the method comprises harvesting a whole blood sample from a living animal, isolating a platelet-rich plasma from the whole blood sample, and adding the platelet-rich plasma to a bioreactor, wherein the bioreactor contains a cell-based meat product. In another embodiment, the method comprises harvesting a whole blood sample from a living animal, isolating a plasma product from the whole blood sample, and adding the plasma product to a bioreactor, wherein the bioreactor contains a cell-based meat product. In some embodiments, cell culture growth media such as described herein can be added to a bioreactor, for example, to stimulate cells with a bioreactor to grow. The bioreactor may be used to produce any of a variety of products, e.g., biopharmaceuticals, pigments, enzymes, etc. As another example, in some embodiments, the bioreactor contains a cultured animal derived meat product. In another example, in some embodiments, the bioreactor is used to produce an alcoholic beverage. In yet another example, the bioreactor processes biomass. Biomass is a renewable organic material that comes from plants and animals. Applications of biomass include, but are not limited to, conversion of bio-feed stock into biofuels, e.g., bioethanol and biobutanol, chemicals, materials, and combustible gases, yeast biomass, etc. In some embodiments, the bioreactor comprises an enzyme. In enzyme bioreactors, enzymes are used to catalyze a biochemical transformation and/or chemical reaction to generate a desired product. In some embodiments, the bioreactor comprises yogurt; in some embodiments, the bioreactor comprises a lactic beverage. In some embodiments, the bioreactor comprises an aroma compound. Aroma compounds are important for food, feed, cosmetic, and pharmaceutical industries. In some embodiments, the bioreactor comprises a pigment. In some embodiments, the bioreactor comprises a protein produced from a bacterial expression system.

In addition, some aspects of the disclosure are directed at sustainably procuring blood components for cultivating products in a bioreactor, for example, such as cell-based meats or biopharmaceuticals. In some embodiments, blood can be collected from non-human animals, for example, to obtain platelets, platelet-rich plasma, etc. For example, repeated blood collection from non-human animals may be used to obtain immune cells, or the like.

As will be appreciated by those skilled in the art, animal derived products, such as plasma, platelets and/or platelet rich plasma (PRP), can be safely harvested, for example, by apheresis, from different animals such as bovines, equines and canines, and used to treat a wide range of injuries in veterinary medicine. Human platelet lysate has also been proposed for cultivation of human stem cells as an animal friendly replacement of FBS. Thus, in some embodiments, plasma, platelets, and platelet-rich plasma may be repeatedly harvested from animals, e.g., without causing health issues to the animals. For example, platelet lysate and platelet rich plasma can be harvested at least 1×, at least 2×, at least 3×, at least 4×, etc., every 30 days, or at other rates such as those described herein. In this way, a cell culture growth medium can be economically produced according to certain embodiments for large scale production of, for example, cultivated meats, without slaughtering the animal and/or without the adverse environmental impacts of raising animals for slaughter.

In some embodiments, apheresis may be used to harvest blood components from the non-human living animal at varying time intervals. As discussed herein, apheresis allows one or more blood components to be selectively removed from the blood and returns the unused blood components back to the animal. Apheresis may be used to selectively harvest plasma, red blood cells, white blood cells, platelet rich plasma, platelet poor plasma, and the like. In some embodiments, apheresis may be used to harvest one or more blood components at spaced intervals without affecting the health of the animal. For example, in some embodiments, apheresis may be used to remove a blood product, such as plasma, at a rate of at least 1% of body weight per blood draw per week, of at least 1.25% of body weight per blood draw per week, of at least 1.5% of body weight per blood draw per week, of at least 1.75% of body weight per blood draw per week, of at least 2% of body weight per blood draw per week, of at least 2.25% of body weight per blood draw per week, of at least 2.5% of body weight per blood draw per week, of at least 2.75% of body weight per blood draw per week, of at least 3.0% of body weight per blood draw per week, or of at least 3.5% of body weight per blood draw per week. In some embodiments, apheresis may be used to remove a blood product, e.g., plasma, at a rate no greater than 3.5% of body weight per blood draw per week, no greater than 3.0% of body weight per blood draw per week, no greater than 2.75% of body weight per blood draw per week, no greater than 2.5% of body weight per blood draw per week, no greater than least 2.25% of body weight per blood draw per week, no greater than 2% of body weight per blood draw per week, no greater than 1.75% of body weight per blood draw per week, no greater than 1.5% of body weight per blood draw per week, no greater than 1.25% of body weight per blood draw per week, no greater than 1% of body weight per blood draw per week.

In other embodiments, apheresis may be used to remove a blood product, e.g., plasma, at a rate of at least 1% of body weight per blood draw per every 2 weeks of at least 1.25% of body weight per blood per every two weeks of at least 1.5% of body weight per blood draw per every two weeks, of at least 1.75% of body weight per blood draw per every two weeks, of at least 2% of body weight per blood draw per every two weeks, of at least 2.25% of body weight per blood draw per every two weeks, of at least 2.5% of body weight per blood draw per every two weeks, of at least 2.75% of body weight per blood draw per every two weeks, of at least 3.0% of body weight per blood draw per every two weeks, or of at least 3.5% of body weight per blood draw per every two weeks. In some embodiments, apheresis may be used to remove a blood product, e.g., plasma, at a rate no greater than 3.5% of body weight per blood draw per every two weeks, no greater than 3.0% of body weight per blood draw per every two weeks, no greater than 2.75% of body weight per blood draw per every two weeks, no greater than 2.5% of body weight per blood draw per every two weeks, no greater than least 2.25% of body weight per blood draw per every two weeks, no greater than 2% of body weight per blood draw per every two weeks, no greater than 1.75% of body weight per blood draw per every two weeks, no greater than 1.5% of body weight per blood draw per every two weeks, no greater than 1.25% of body weight per blood draw per every two weeks, no greater than 1% of body weight per blood draw per every two weeks.

Fluid replacement therapy may be used for larger blood draws. The frequency of blood sampling may be dependent on the total volume. In some embodiments, a single blood draw may comprise up to 15% of the blood volume without fluid therapy and up to 20% if followed by fluid therapy within a two-week period. Fluid therapy helps to replace the lost blood volume and may be used prevent the animal from going into hypovolemic shock. Any suitable fluid known to those of skill in the art may be used, including saline, Plasma-Lyte, dextran40, and the like. Accordingly, in one set of embodiments, one or more blood draws may be withdrawn or taken from a non-human animal, e.g., to produce a plasma product, a platelet lysate (PL) and/or platelet rich plasma (PRP), e.g., including those described herein. Exemplary embodiments of non-human animals include chicken, cow, pig, mutton, goat, deer, fish, duck, turkey, shrimp, or other animals that are commonly recognized for widespread human consumption. The blood may be processed in some cases to isolate the various components, e.g., plasma, platelet-rich plasma, red blood cells etc., using any suitable technique, e.g., centrifugation and apheresis. For example, apheresis can be used to separate blood components by passing blood through a machine programmed to remove a particular blood component, e.g., platelets, immune cells, etc. In some cases, the unused blood components may be returned to the animal, which permits more frequent donation of blood components that are rapidly replenished in vivo, e.g., blood platelets. In some cases, blood samples can be withdrawn and processed, e.g., by centrifugation, to separate the platelet-rich plasma, immune cells (i.e., the buffy coat), red blood cells, etc.

In some cases, blood may be withdrawn from a donor animal at spaced intervals, which may be regular or irregular. Between blood draws, the animal can recover and produce new blood. Any suitable interval may be used. For example, the blood may be withdrawn from the animal every 2 weeks, every 4 weeks, every 6 weeks, every 2 months, or the like. As an example, in some embodiments, a blood draw may be withdrawn from an animal, and after a suitable interval, an additional blood draw may be withdrawn from the animal. This cycle can also be repeated any suitable number of times. The blood draws may each be processed, for example, as discussed herein. For example, the blood may be used to isolate platelets and/or platelet-rich plasma.

In addition, the animal may also be subjected to second, third, etc. blood draws, e.g., at spaced intervals such as discussed herein. The blood withdrawn in each draw may be treated in the same way, or in different ways, depending on the application. In some embodiments, the animal remains alive between blood draws, and can recover and produce new blood. In this way, although blood production from the animal occurs, the animal is not slaughtered in order for blood production from the animal to continue.

Additionally, in some cases, any of the cells and blood components described herein can be isolated from the blood of slaughtered animals.

It is generally believed that in accordance with some embodiments, substituting platelet lysate (PL) and platelet rich plasma (PRP) for fetal bovine serum, as the primary source of growth factors for cell culture media may reduce the economic burden of large-scale production of cultivated meats, making the cost of production similar to traditional meat products, albeit with a lower environmental impact and without the adverse effects to the animals. For example, according to one calculation, a single cow, which lives for about 15 to 20 years, can provide sufficient blood products to produce enough cultivated meat equal to 50 cattle raised for slaughter. Thus, the practices described in certain embodiments of the current disclosure may be able to substantially reduce the environmental impact of traditional agricultural practice.

In addition, some aspects of the disclosure relate to cell-based meat products, e.g., that are grown or produced in bioreactors using cell culture growth media such as is described herein. In some embodiments, the cell-based meat product comprises non-human cells, for example, platelets, stem cells, myoblasts, myotubes, fibroblasts, adipose cells, etc. The non-human cells may arise from any suitable source, including cow, pig, sheep, goat, deer, fish, duck, turkey, shrimp, or any other animal that is commonly recognized for widespread human consumption. In some embodiments, the cell-based meat product comprises a muscle replica, such as described in U.S. Ser. No. 63/279,617, filed Nov. 15, 2021, entitled “Constructs Comprising Fibrin or Other Blood Products for Meat Cultivation and Other Applications,” by Hosseini, et al., incorporated herein by reference in its entirety. A muscle replica may comprise muscle cells, e.g., myoblasts, that are cultured on a microcarrier, or other substrate, such as a fibrin microcarrier, in a bioreactor, or other cell-culture system. In some embodiments, the myoblasts may fuse together to form myotubes, which are the foundation of muscle fibers and meat in general, to yield a product that resembles ground beef. In addition, in some cases, the microcarriers or scaffolds may have structures, such as grooves, that may allow the cells such as myoblasts to become aligned in a specific direction, although this is not a requirement. Such structures are described in U.S. Ser. No. 63/159,403, filed Mar. 10, 2021, entitled “Constructs for Meat Cultivation and Other Applications,” by Khademhosseini, et al., incorporated herein by reference in its entirety.

In some embodiments, the cell-based meat product comprises a fat replica, to improve the appearance, taste, and texture of the meat product, such as described in U.S. Ser. No. 63/279,642, filed Nov. 15, 2021, entitled “Systems and Methods of Producing Fat Tissue for Cell-Based Meat Products,” by Hosseini, et al., incorporated herein by reference in its entirety. For instance, a fat replica can be formed by dispersing a fat, e.g., any animal or plant oil, e.g., sunflower oil, or animal-based cell, e.g., adipocytes, in a non-human blood plasma, then causing the blood plasma to crosslink and/or clot, e.g., forming a hydrogel containing the fat dispersion. In other embodiments, fat cells, e.g., adipocytes or adipose progenitor cells, can be seeded on a microcarrier, or other edible scaffolding material, and allowed to grow, e.g., in a cell culture system such as a bioreactor to yield a fat replica that may be mixed with the muscle replica.

In some embodiments, the cell-based meat product comprises a lysate of red blood cell to impart coloring or “redness” into the meat product, such as described in U.S. Ser. No. 63/279,644, filed Nov. 15, 2021, entitled “Production of Heme for Cell-Based Meat Products,” by Hosseini, et al., incorporated herein by reference in its entirety. Thus, some embodiments are directed toward a lysate of red blood cells. As described herein, such a lysate may be produced, for example, by lysing red blood cells, using any of a variety of lysing techniques, e.g., exposure to hypoosmotic water, acoustic cavitation, or extrusion through a membrane.

The muscle replica, fat replica, lysate of red blood cell, and platelets, may be present in any suitable amount with the cell-based meat product. For example, the muscle replica may be present at least 10 wt %, at least 20 wt %, at least 30 wt %, at least 40 wt %, at least 50 wt %, at least 60 wt %, at least 70 wt %, at least 80 wt %, at least 90 wt %, etc., and/or no more than 95 wt %, no more than 90 wt %, no more than 80 wt %, no more than 70 wt %, no more than 60 wt %, no more than 50 wt %, no more than 40 wt %, no more than 30 wt %, no more than 20 wt %, no more than 10 wt %, etc. Similarly, the fat replica may be present at least 10 wt %, at least 20 wt %, at least 30 wt %, at least 40 wt %, at least 50 wt %, at least 60 wt %, at least 70 wt %, at least 80 wt %, at least 90 wt %, etc., and/or no more than 95 wt %, no more than 90 wt %, no more than 80 wt %, no more than 70 wt %, no more than 60 wt %, no more than 50 wt %, no more than 40 wt %, no more than 30 wt %, no more than 20 wt %, no more than 10 wt %, etc. The cell lysate may also be present at least 10 wt %, at least 20 wt %, at least 30 wt %, at least 40 wt %, at least 50 wt %, at least 60 wt %, at least 70 wt %, at least 80 wt %, at least 90 wt %, etc., and/or no more than 95 wt %, no more than 90 wt %, no more than 80 wt %, no more than 70 wt %, no more than 60 wt %, no more than 50 wt %, no more than 40 wt %, no more than 30 wt %, no more than 20 wt %, no more than 10 wt %, etc.

In some embodiments, the platelet-rich plasma comprises at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, at least 5%, at least 6%, at least 7%, at least 8%, at least 9%, at least 10%, etc., of the cells in the final cell-based meat product, and/or no more than 10%, no more than 9%, no more than 8%, no more than 7%, no more than 6%, no more than 5%, no more than 4%, no more than 3%, no more than 2%, no more than 1%, no more than 0.9%, no more than 0.8%, no more than 0.7%, no more than 0.6%, no more than 0.5%, no more than 0.4%, no more than 0.3%, no more than 0.2%, no more than 0.1%, etc., of the cells in the final cell-based meat product. In some embodiments, at least 0.1% of cells in the cell-based meat product are the platelet rich plasma. In some embodiments, the platelet-rich plasma comprises bovine platelet rich plasma.

In some embodiments, the cell-based meat product comprises a platelet lysate. As described herein, such a lysate may be produced, for example, by lysing blood platelets, using any of a variety of lysing techniques, e.g., exposure to hypoosmotic water, acoustic cavitation, or extrusion through a membrane. In some embodiments, the platelet lysate comprises at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, at least 5%, at least 6%, at least 7%, at least 8%, at least 9%, at least 10%, etc., of the cells in the final cell-based meat product, and/or no more than 10%, no more than 9%, no more than 8%, no more than 7%, no more than 6%, no more than 5%, no more than 4%, no more than 3%, no more than 2%, no more than 1%, no more than 0.9%, no more than 0.8%, no more than 0.7%, no more than 0.6%, no more than 0.5%, no more than 0.4%, no more than 0.3%, no more than 0.2%, no more than 0.1%, etc., of the cells in the final cell-based meat product. In some embodiments, at least 0.1% of cells in the cell-based meat product are the platelet lysate. In some embodiments, the platelet lysate comprises platelet lysate.

In some embodiments, the cell-based meat product comprises a platelet lysate and a plasma product, wherein at least 0.1% of the cells in the cell-based meat product are platelet cells. In another embodiment, the cell-based meat product comprises a platelet-rich plasma and a plasma product, wherein at least 0.1% of the cells in the cell-based meat product are platelet cells. In yet another embodiment, the cell-based meat product comprises a platelet-rich plasma and a platelet lysate, wherein at least 0.1% of the cells in the cell-based meat product are platelet cells.

In addition, some aspects of the disclosure relate to producing a cultivated product, e.g., products that are grown or produced in bioreactors using cell culture growth media such as is described herein. In some embodiments, the cultivated product comprises a leather product that could be used, for example, to produce clothing and textiles. Other non-limiting examples of cultivated products include milk, fur, hair, wool, organs, horn, tusk, or the like.

The following are each incorporated herein by reference in their entireties: U.S. Provisional Patent Application Ser. No. 63/159,403, filed Mar. 10, 2021, entitled “Constructs for Meat Cultivation and Other Applications”; U.S. Provisional Patent Application Ser. No. 63/279,617, filed Nov. 15, 2021, entitled “Constructs Comprising Fibrin or Other Blood Products for Meat Cultivation and Other Applications”; U.S. Provisional Patent Application Ser. No. 63/279,631, filed Nov. 15, 2021, entitled, “Methods and Systems of Preparing Cultivated Meat from Blood or Cellular Biomass”; U.S. Provisional Patent Application Ser. No. 63/279,642, filed Nov. 15, 2021, entitled, “Systems and Methods of Producing Fat Tissue for Cell-Based Meat Products”; U.S. Provisional Patent Application Ser. No. 63/279,644, filed Nov. 15, 2021, entitled “Production of Heme for Cell-Based Meat Products”; US Provisional Patent Application Ser. No. U.S. 63/300,577, filed Jan. 18, 2022, entitled “Animal-Derived Antimicrobial Systems and Methods”; U.S. Provisional Patent Application Ser. No. 63/164,397, filed Mar. 22, 2021, entitled “Growth Factor for Laboratory Grown Meat”; U.S. Provisional Patent Application Ser. No. 63/164,387, filed Mar. 22, 2021, entitled, “Methods of Producing Animal Derived Products”; US Provisional Patent Application Ser. No. 63/314,171, filed Feb. 25, 2022, entitled “Growth Factors for Laboratory Grown Meat and Other Applications”; and US Provisional Patent Application Ser. No. 63/314,191, filed Feb. 25, 2022, entitled “Methods and Systems of Producing Products Such as Animal Derived Products.”

The following examples are intended to illustrate certain embodiments of the present disclosure, but do not exemplify the full scope of the disclosure.

### Example 1

According to this example, the effects of human Platelet Lysate (hPL) on lamb myoblast proliferation was evaluated at concentrations of 0.5%, 1% and 2%, and the combination of 2% hPL with 8% Bovine Plasma and were compared against a commercially available media formula known for culturing of cultivated meat Essential 8™ (Thermo Fisher Scientific) and with a basic serum free cell culture medium (Dulbecco's modified Eagle Medium (DMEM), GlutaMAX™, and Sodium Pyrovate from Gibco) without supplement used as control. Primary myoblasts isolated from lamb were cultured for 5 days at initial density of 4000 cell per square centimeter in 96 well plates with the resulting cell counts shown in FIG. 1. These results show that the combination of 2% human platelet lysate (hPL) with 8% bovine plasma was the most effective formula on cell proliferation.

### Example 2

According to this example, the effects of various media compositions were evaluated for their effects on the growth of primary lamb myoblasts. Lamb myoblasts were cultured for 5 days at initial density of 4000 cell per square centimeter in 96 well plates with the media exchanged every other day. The following media formulas were tested: (1) A Basic serum free media (DMEM, Glutamax, Pyrovate from Gibco) without supplement was used as control; (2) 2% bovine Platelet Lysate (bPL) with an initial platelet count of 2×10⁹/ml): (3) 2% bovine Platelet Lysate (bPL) with an initial platelet count of 0.2×10⁹/ml; (4) bovine platelet poor plasma from citrated blood (bPP Plasma-1); (5) bovine platelet poor plasma from defibrinated blood bPP (Plasma-2); (6) bovine platelet rich plasma (obtained by 25 min centrifugation at 300 g) (bPRP) with the platelets lysed by freeze-thaw: (7) human platelet lysate (hPL) from a commercial vendor (Stem Cell Technologies, US); and (8) FBS: fetal bovine serum (Invitrogen, US). The resulting cell counts are shown in FIG. 2A and cell doubling time is shown in FIG. 2B for each medium. These results show that the growth results for bovine Platelet Rich Plasma were statistically equivalent to those with Fetal Bovine Serum and substantially better than the control and remaining growth media.

### Example 3

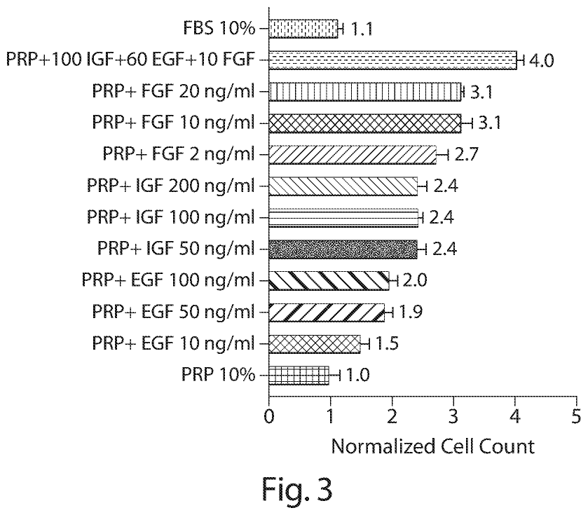

This example demonstrates the ability of an improved cell culture growth media, comprising platelet-rich plasma, to enhance bovine myoblast proliferation, compared to fetal bovine serum. Bovine myoblasts were suspended in DMEM containing 10% fetal bovine serum and seeded at a cell density of 10,000 cells/well to allow for adhesion to the culture substrate. After 6 hours, the culture media was removed and fresh DMEM (serum free) was added. After 12 hours, the cell culture media was again removed and replaced with DMEM containing 10% bovine platelet-rich plasma. The cells were subsequently incubated for 24 hours at 37° C. in 95% O₂ and 5% CO₂, after which, the cell culture medium was removed. In a subset of experiments, the bovine platelet-rich plasma was supplemented with increasing concentrations of epidermal growth factor (EFG, 10 ng/mL, 50 ng/ml, or 100 ng/ml), insulin growth factor (IGF, 50 ng/mL, 100 ng/mL, or 200 ng/mL), fibroblast growth factor (FGF, 2 ng/mL, 10 ng/ml, or 20 ng/mL), or a combination of IGF (100 ng/mL), EGF (50 ng/mL), and FGF (10 ng/mL). The cultured cells were then washed 3× with phosphate buffered saline to remove all media, trypinsized to release them from the culture substrate, and the cell numbers determined using optical techniques. The results indicated that platelet-rich plasma was equally as effective as fetal bovine serum at stimulating myoblast proliferation. The results also demonstrated that supplementing the platelet-rich plasma with additional growth factors increased cell proliferation in a concentration dependent manner (FIG. 3).

### Example 4

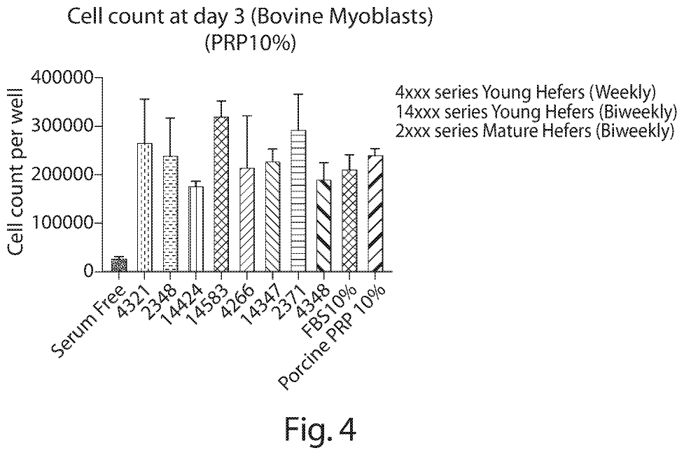

This example demonstrates the performance of platelet-rich plasma isolates from different cows. Table 1 contains information related to the types of cows used, their date of birth, weight, total volume of donated platelet-rich plasma, and donation frequency. The platelet-rich plasma from each animal was then taken and used to produce a cell-culture growth medium. To determine the proliferative potential of such solutions, bovine myoblasts were suspended in DMEM containing 10% fetal bovine serum and seeded at a cell density of 10,000 cells/well to allow for adhesion to the culture substrate. After 6 hours, the culture media was removed and fresh DMEM (serum free) was added. After 12 hours, the cell culture media was again removed and replaced with DMEM containing 10% bovine platelet-rich plasma isolates from the various cows. Control samples were incubated with 10% fetal bovine serum or 10% porcine platelet-rich plasma (obtained from a slaughterhouse). The cells were subsequently incubated for 24 hours at 37° C. in 95% O₂ and 5% CO₂, after which, the cell culture medium was removed. The cultured cells were then washed 3× with phosphate buffered saline to remove all media, trypsinized to release them from the culture substrate, and the cell numbers determined using optical techniques. The results indicated that the platelet-rich plasma, isolated from the bovine donors, significantly enhanced cell proliferation, relative to serum free media, and was equally effective as fetal bovine serum, regardless of the donor cow (FIG. 4). In addition, Table 1 shows information related to the types of cows used, their date of birth, weight, volume of donated platelet-rich plasma, and donation frequency.

TABLE 1
Young Holstein Hefer

| | DOB | Weight (lb) | Donated PRP (L) |
|---|---|---|---|
| 4266 | Jun. 18, 2019 | 1350 | 9.8 |
| 4321 | Jul. 19, 2019 | 1450 | 10.5 |
| 4348 | Jul. 31, 2019 | 1450 | 10.5 |

Donation frequency: Weekly

| | | | |
|---|---|---|---|
| Number | DOB | Weight (lb) | PRP (L) |

Mature Holstein Cow

| | | | |
|---|---|---|---|
| 2317 | Jul. 17, 2016 | 1950 | 14.2 |
| 2348 | | 1950 | 14.2 |
| 2315 | | 1950 | 13.8 |

Donation frequency: Biweekly

Young Holstein Hefer

| | | | |
|---|---|---|---|
| 14347 | Jul. 24, 2019 | 1250 | 9.1 |
| 14424 | Aug. 10, 2019 | 1250 | 9.1 |
| 14583 | Sep. 26, 2019 | 1250 | 9.1 |

Donation frequency: Biweekly

Young Steer

| | | | |
|---|---|---|---|
| 5760 | Sep. 27, 2019 | 1400 | 10.2 |
| 5726 | Sep. 22, 2019 | 1350 | 9.8 |
| 5211 | May 26, 2019 | 1330 | 9.7 |

Donation frequency: Biweekly

### Example 5

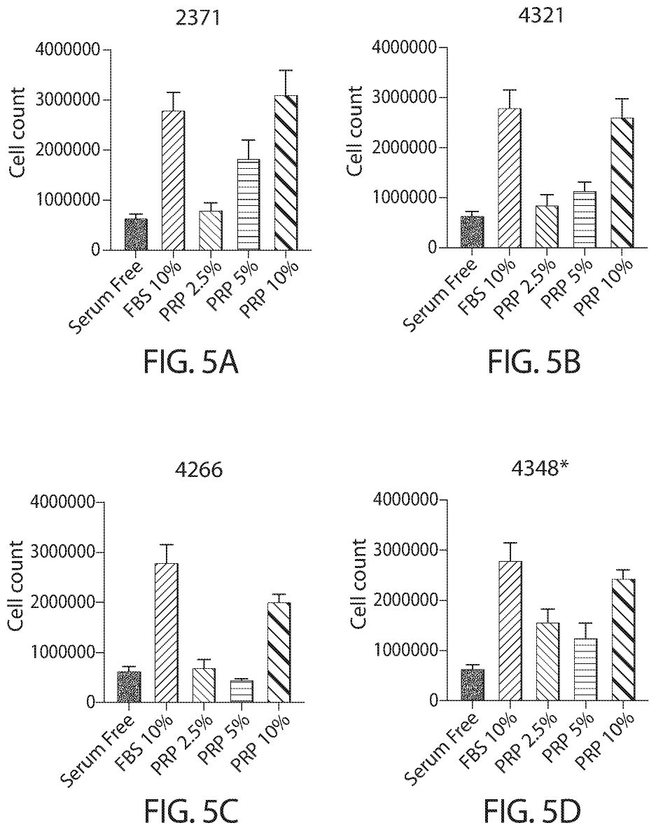

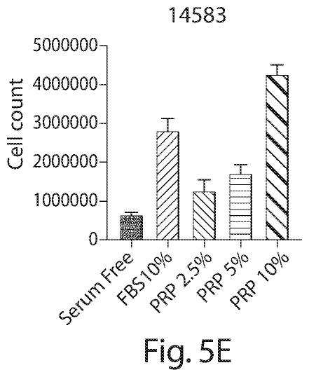

This example demonstrates the effect of varying the platelet-rich plasma concentration, isolated from various donor cows (see Table 1) on bovine myoblast proliferation. Platelet-rich plasma from a mature Holstein Cow (2371) and several young Holstein Heifers (4321, 4266, 4348, and 14583) was obtained and added to DMEM at a final concentration of 2.5 wt %, 5 wt %, or 10 wt %. To determine the proliferative potential of such solutions, bovine myoblasts were suspended in DMEM containing 10% fetal bovine serum and seeded at a cell density of 10,000 cells/well to allow for adhesion to the culture substrate. After 6 hours, the culture media was removed and fresh DMEM (serum free) was added. After 12 hours, the cell culture media was again removed and replaced with DMEM containing the varying concentrations of bovine platelet-rich plasma (as described above). Control samples were incubated in serum free DMEM or DMEM containing 10% fetal bovine serum. The cells were subsequently incubated for 24 hours at 37° C. in 95% O₂ and 5% CO₂, after which, the cell culture medium was removed. The cultured cells were then washed 3× with phosphate buffered saline to remove all media, trypsinized to release them from the culture substrate, and the cell numbers determined using optical techniques. The results showed that cell proliferation increased with increasing concentrations of platelet-rich plasma; and that 10% platelet-rich plasma was as effective as 10% fetal bovine serum at promoting cell proliferation (FIG. 5).

### Example 6

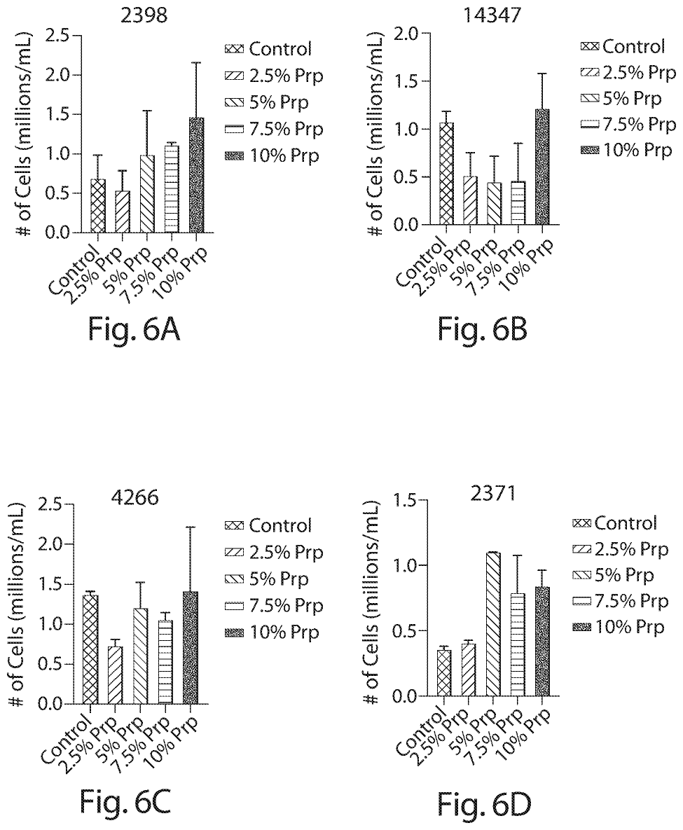

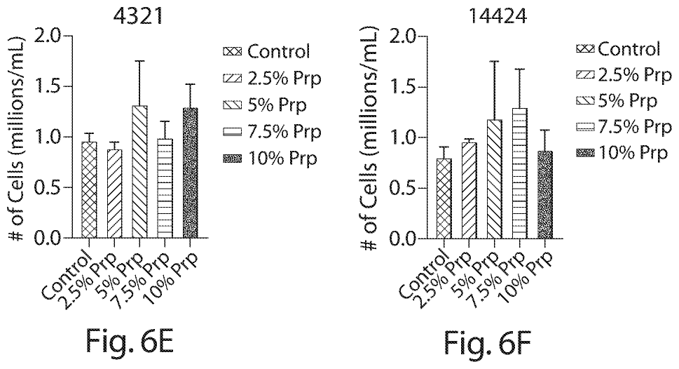

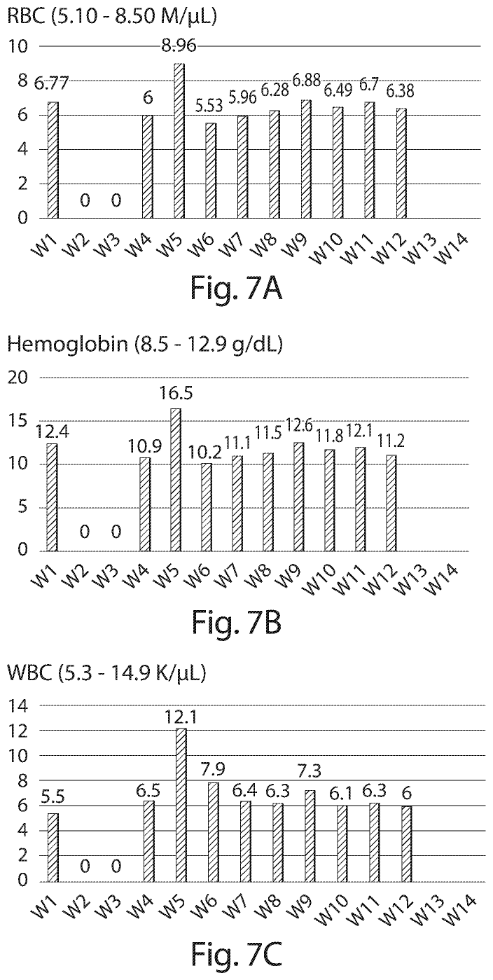

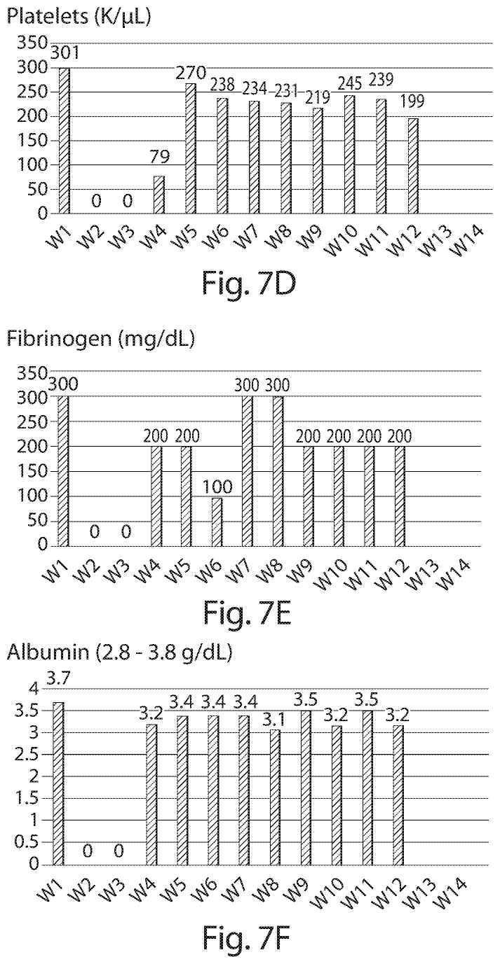

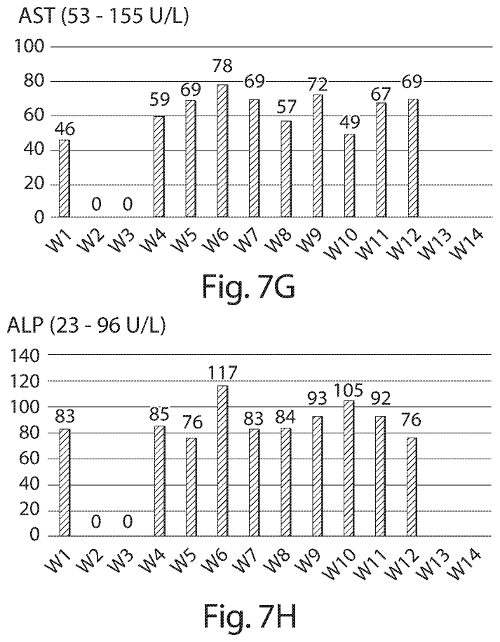

This example demonstrates the effect of varying the platelet-rich plasma concentration, isolated from various donor cows (see Table 1) on human hepatocyte proliferation. Platelet-rich plasma from a mature Holstein Cow (2371) and several young Holstein Heifers (4321, 4266, 4348, and 14583) was obtained and added to DMEM at a final concentration of 2.5 wt %, 5 wt %, 7.5 wt % or 10 wt %. To determine the proliferative potential of such solutions, human hepatocytes were suspended in DMEM containing 10% fetal bovine serum and seeded at a cell density of 10,000 cells/well to allow for adhesion to the culture substrate. After 6 hours, the culture media was removed and fresh DMEM (serum frec) was added. After 12 hours, the cell culture media was again removed and replaced with DMEM containing the varying concentrations of bovine platelet-rich plasma (as described above). Control samples were incubated in serum free DMEM. The cells were subsequently incubated for 24 hours at 37° C. in 95% O₂ and 5% CO₂, after which, the cell culture medium was removed. The cultured cells were then washed 3× with phosphate buffered saline to remove all media, trypsinized to release them from the culture substrate, and the cell numbers determined using optical techniques. The results showed that cell proliferation increased with increasing concentrations of platelet-rich plasma with 10% platelet-rich plasma being the most effective (FIG. 6).

### Example 7

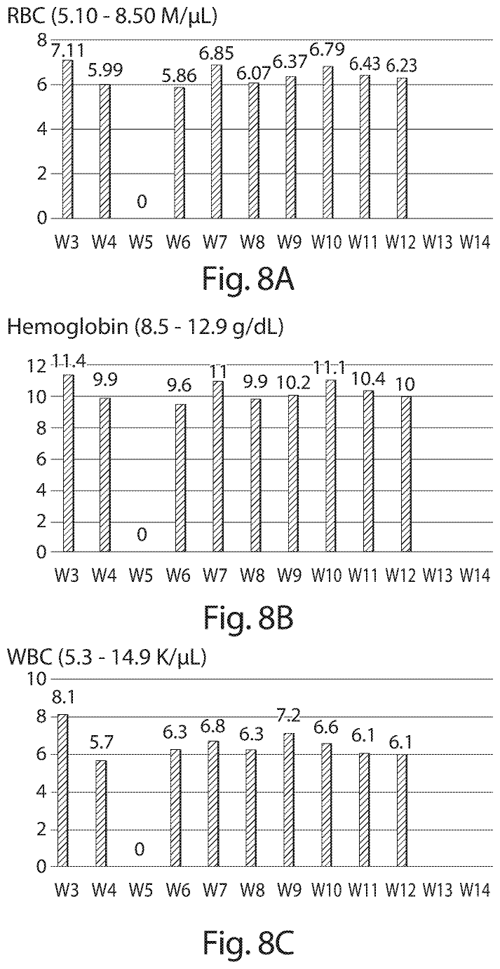

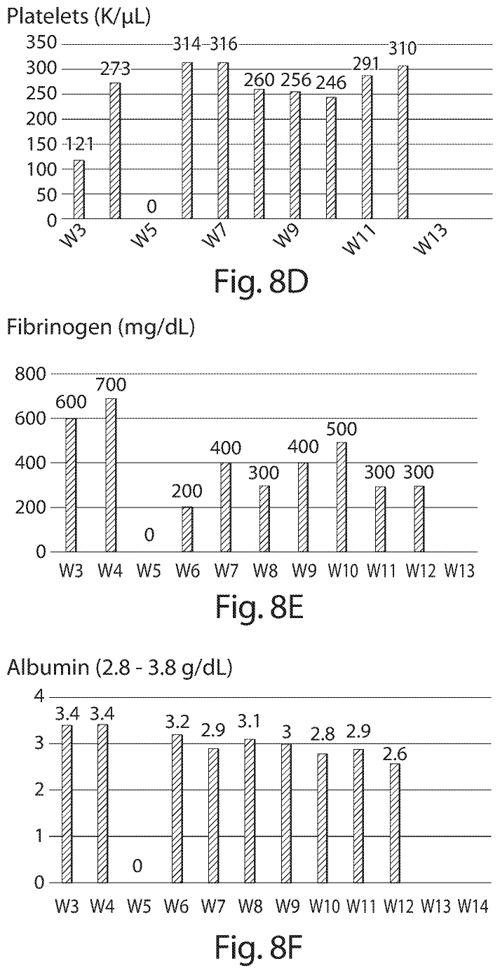

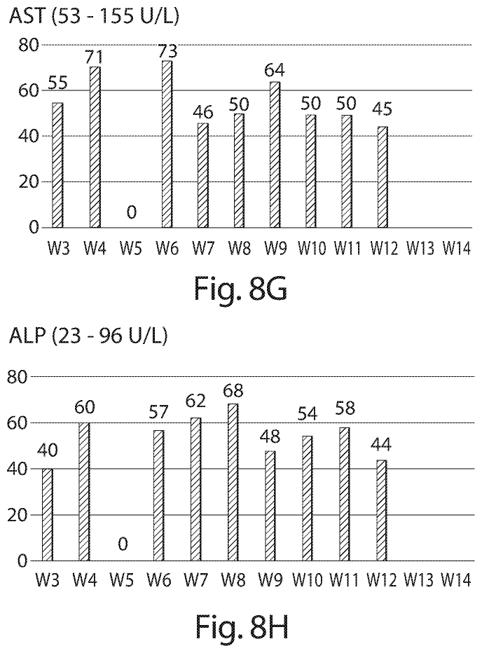

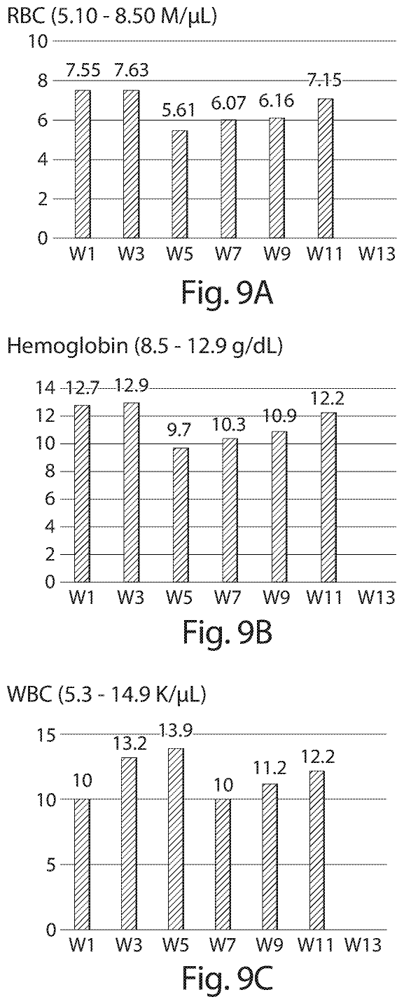

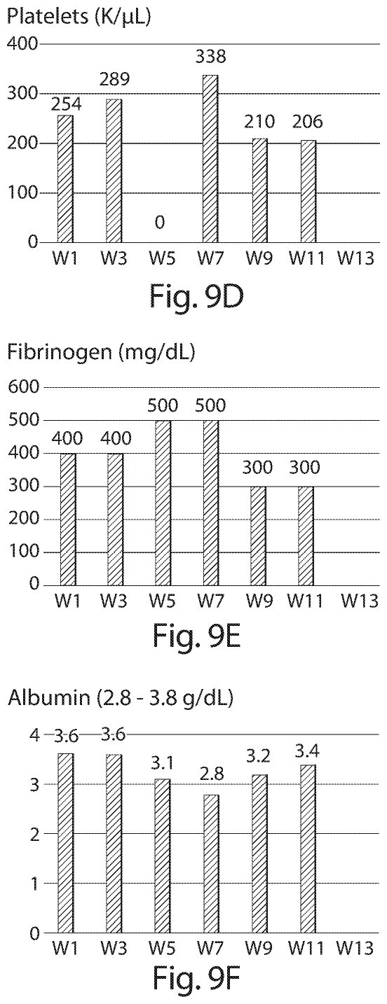

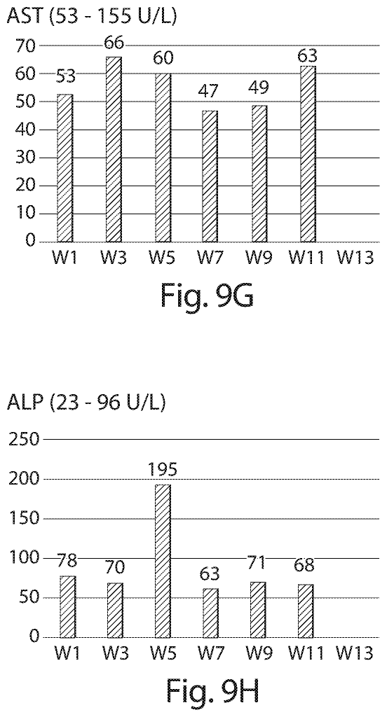

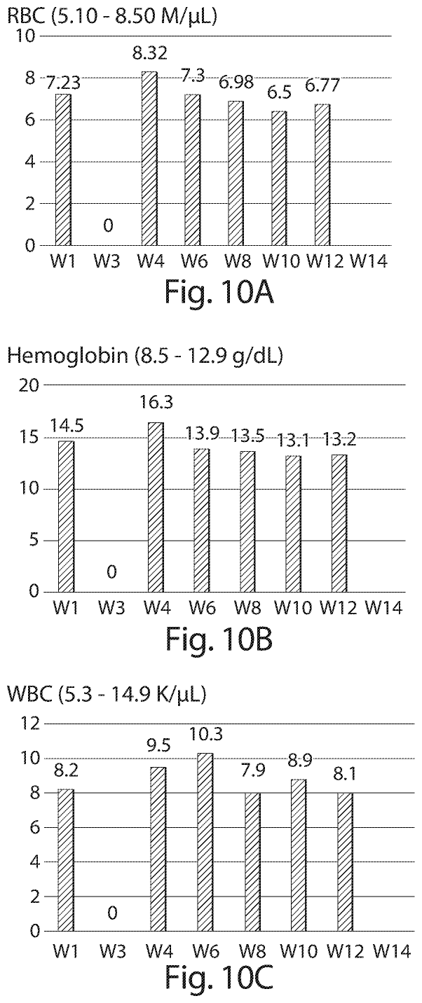

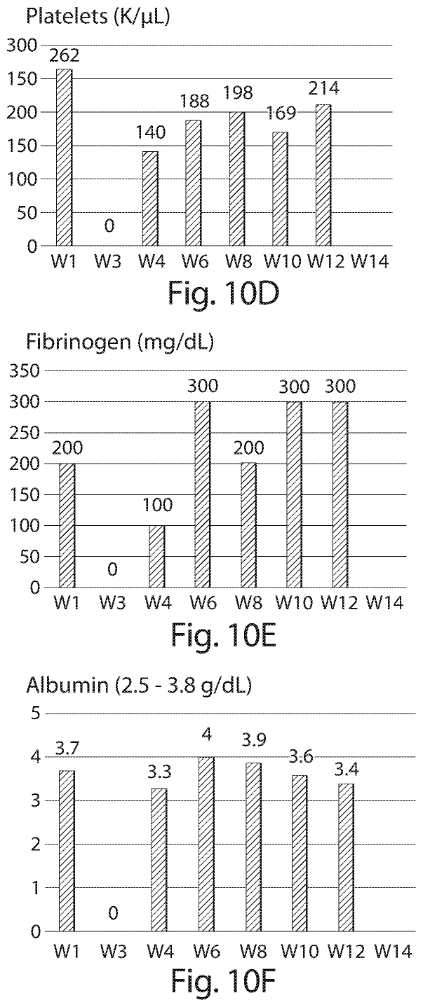

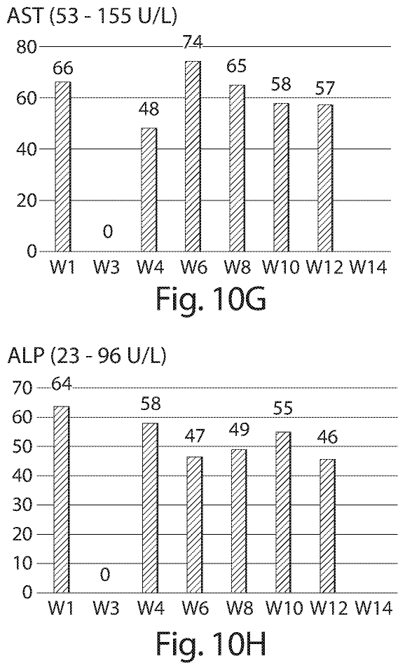

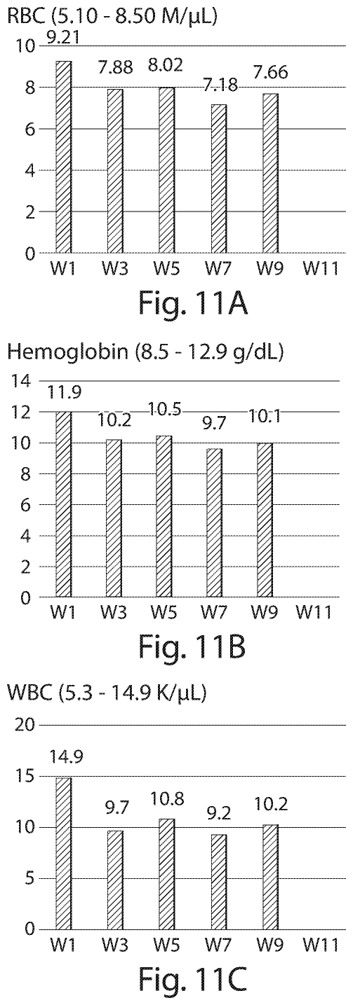

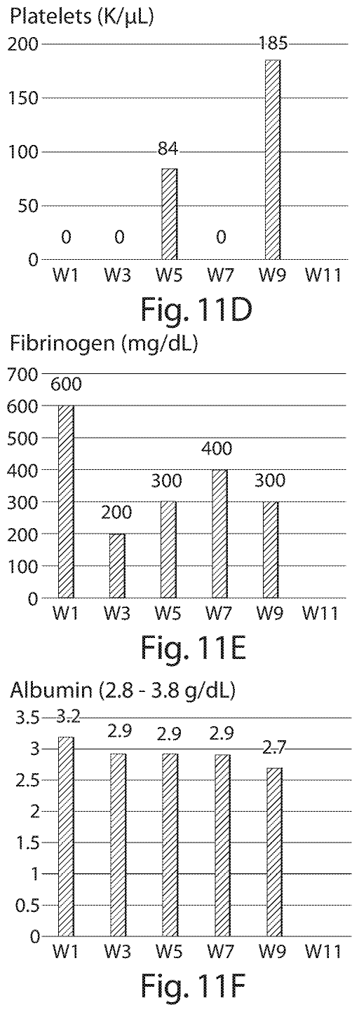

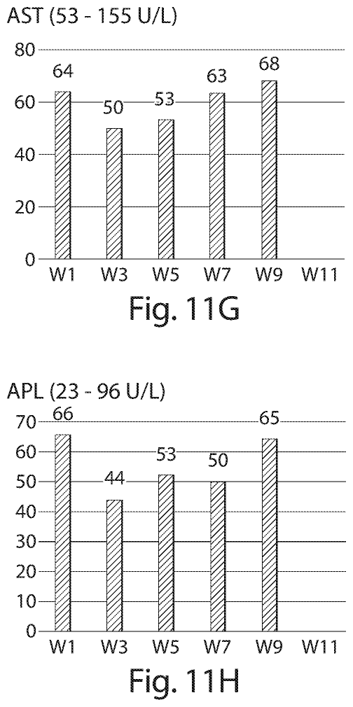

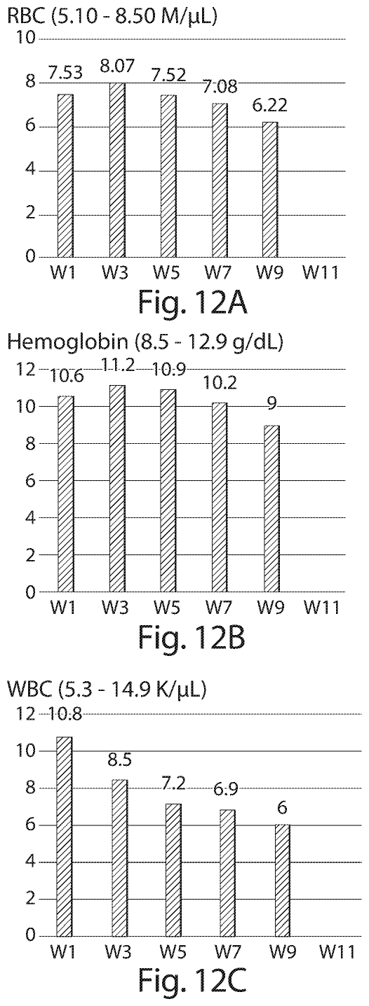

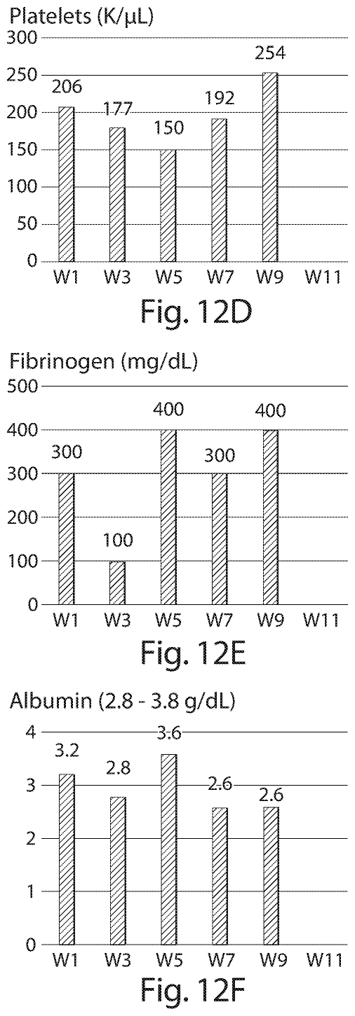

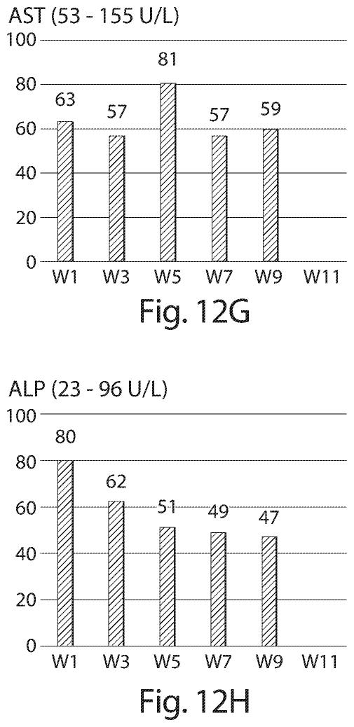

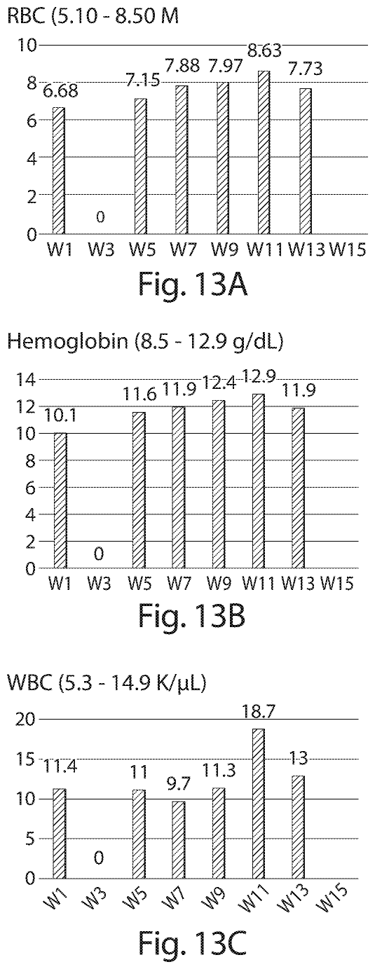

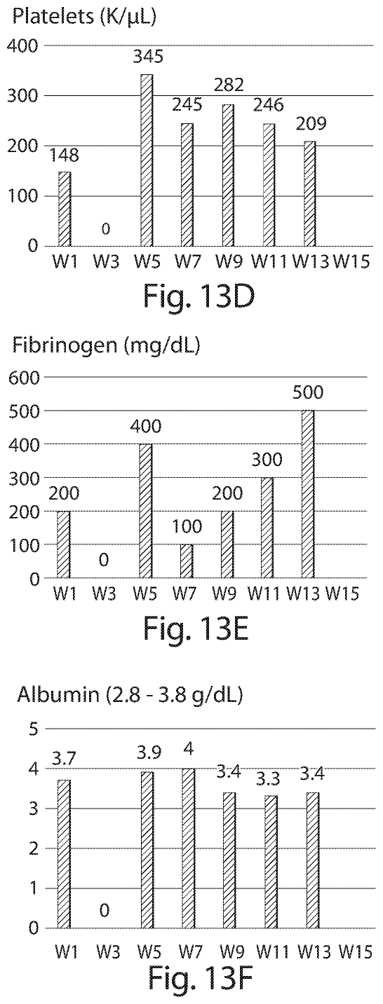

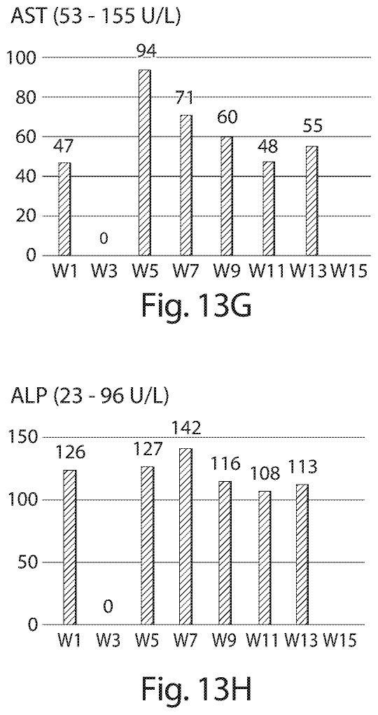

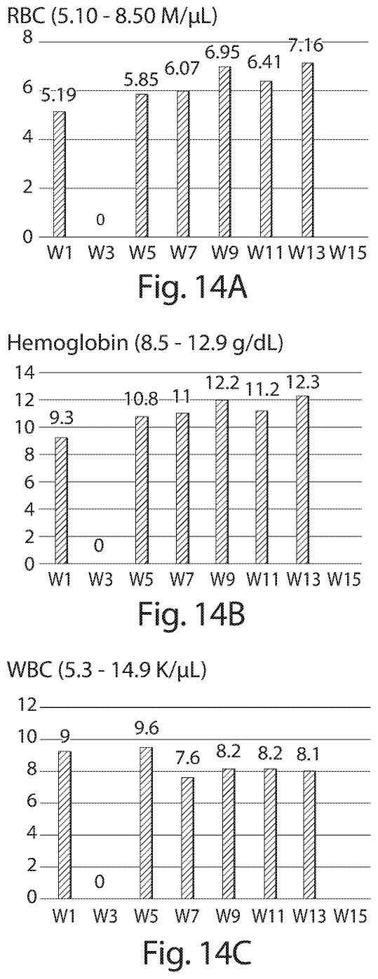

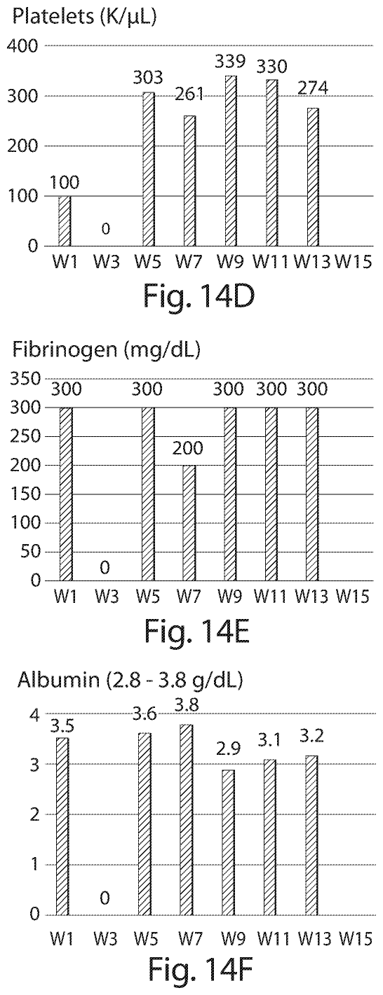

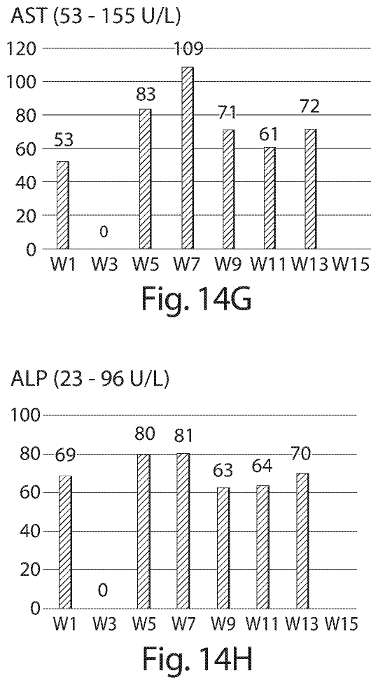

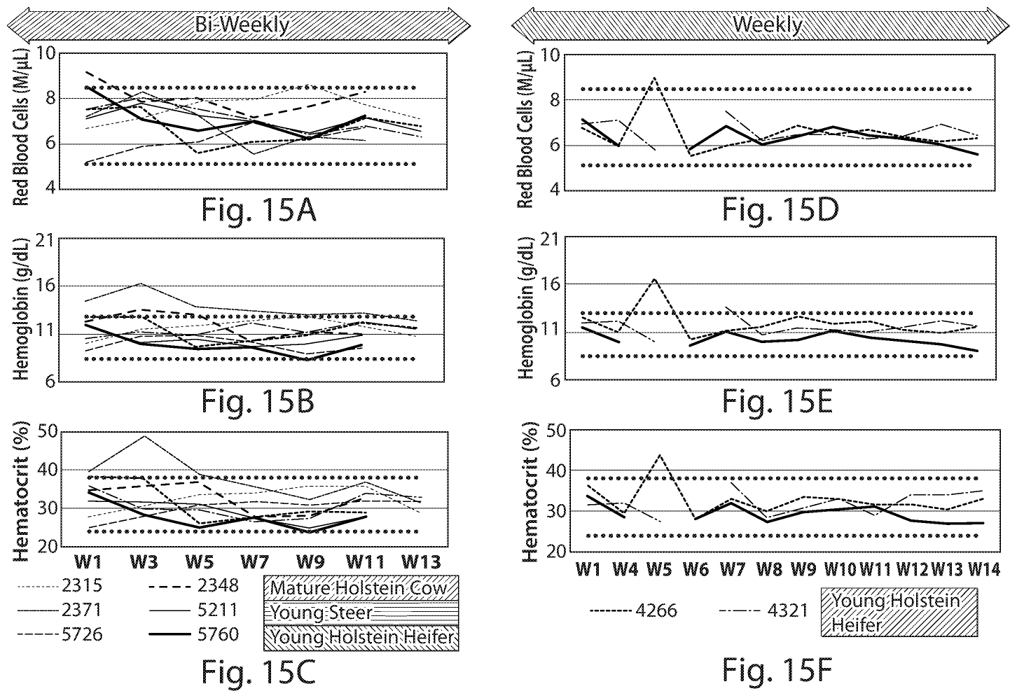

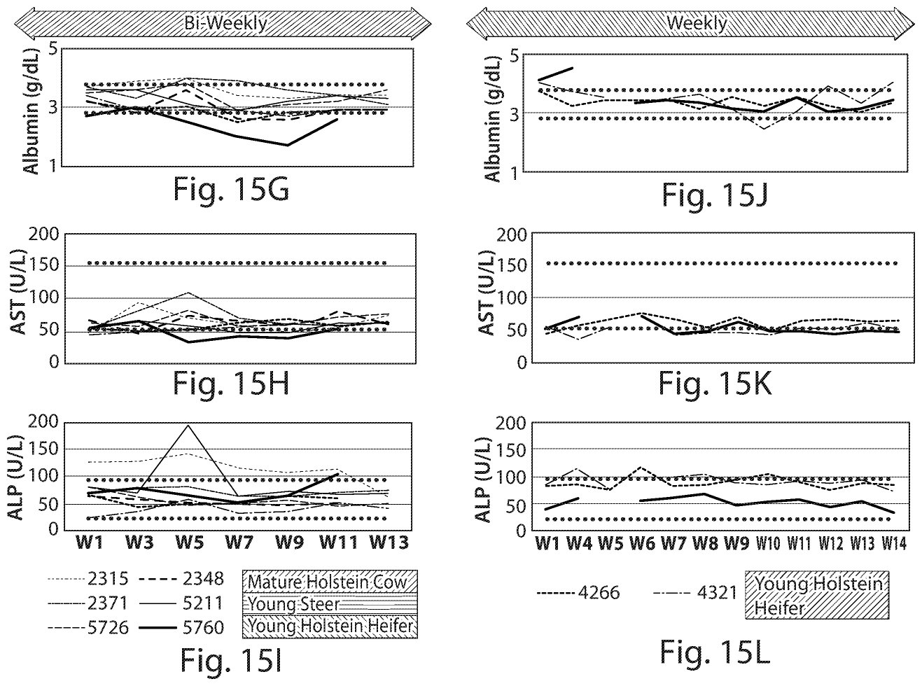

This example demonstrates the effect of repeated blood draws on the heath of the animal donors (see Table 1). Platelet-rich plasma was withdrawn either weekly on young Holstein heifers (4266, 4348) or biweekly on mature Holstein cows (2315, 2371), young steers (5211, 5276), and young Holstein heifers (14424 and 14583) for 9 weeks, 12 weeks, or 13 weeks. The total volume of platelet-rich plasma donated varied from 9 L to 14 L (for reference, the total blood volume of a cow is ˜55 mL/kg or about 33 L for a cow that weighs 1350 pounds). Whole blood draws were performed at the time of platelet-rich plasma donation and standard hematology and blood chemistry was performed. These tests are routine in veterinary medicine and provide information on the health of the animal. The tests report the red blood cell count (e.g., metric for anemia), hemoglobin levels (e.g., metric for anemia), white blood cell count (e.g., metric of infection), platelet counts (e.g., metric of clotting disorders), fibrinogen concentrations (e.g., metric of clotting disorders), albumin levels (e.g. metric of liver health), AST levels (e.g. metric of liver damage) and ALP levels (e.g. metric of liver damage). As can be seen in FIG. 8-15, there are no observed adverse health effects from repeated platelet-rich plasma donation from young or mature Holstein cows and young steers.

### Example 8

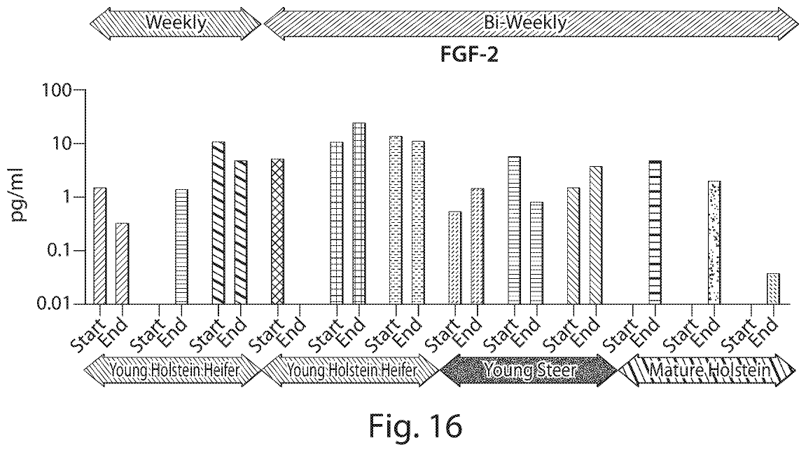

This example illustrates experiments using the apheresis process on cows to collect platelet rich plasma at the volume equal to 1.6% of the cow weight in weekly and biweekly regimes for durations of up to 3 months. The study results are shown in FIG. 16 for bi-weekly (FIGS. 16A-16F) and weekly (FIGS. 16G-16L) blood draws.

This study showed that weekly collection relative to biweekly collection did not affect the major blood and liver marker related to health of the cows. In these figures, the dashed lines show the upper and lower limit of specified markers that are routinely checked from clinical perspective, including red blood cells, hemoglobin, and hematocrit.

### Example 9

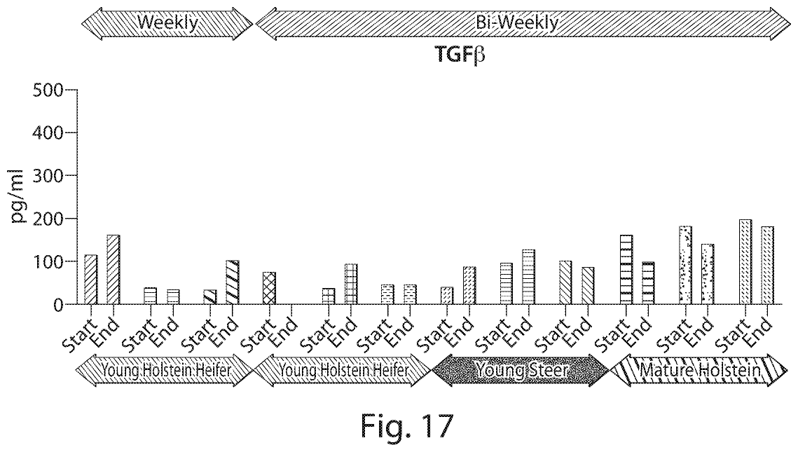

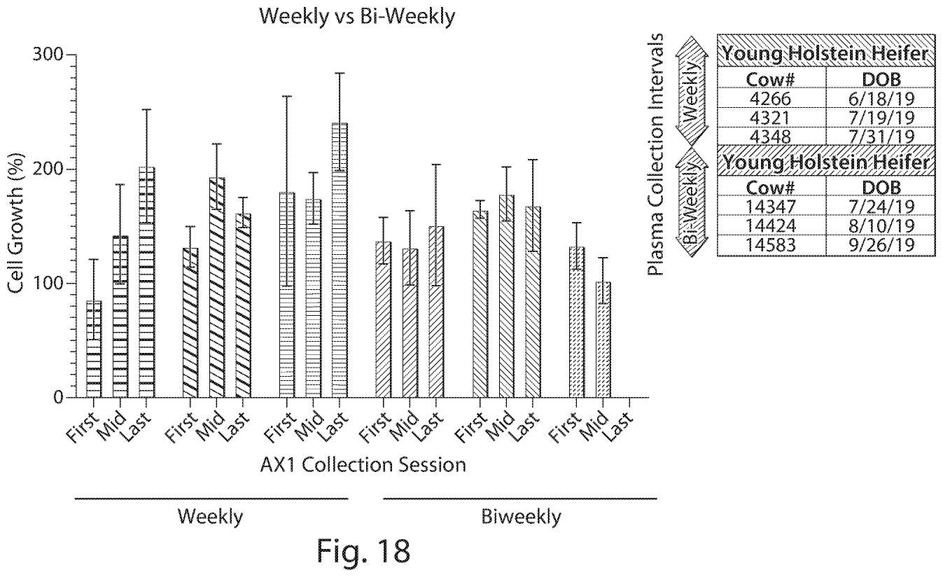

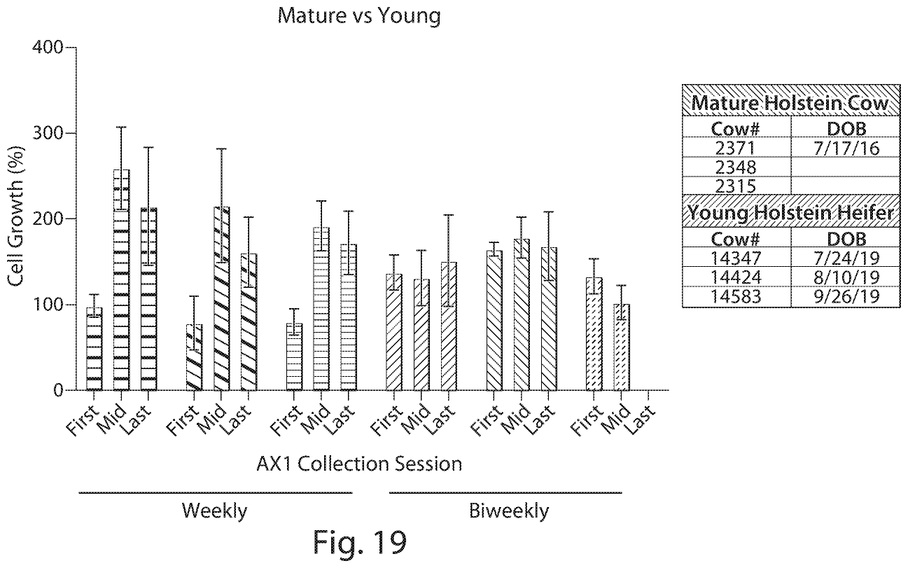

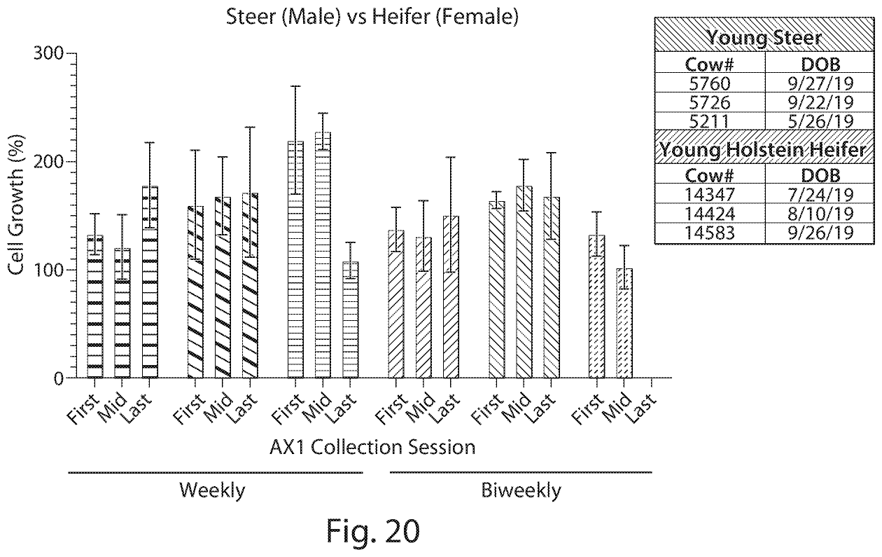

This example illustrates the effect of repeated blood draws on the blood concentration levels of basic fibroblast growth factor-2 (FGF-2) and transforming growth factor beta (TGF-beta) in a. Platelet-rich plasma was extracted from a young Holstein Heifer, a young Steer, and a mature Holstein either weekly or biweekly for 13 weeks using apheresis. The concentration of FGF-2 and TGF-beta in the platelet-rich plasma was determined using standard enzyme-linked immune-absorbent assays. Repeated blood draws had no effect on the plasma concentration levels of either growth factor (FIG. 16 and FIG. 17). Interestingly, a retrospective analysis showed that the concentration of growth factor appeared to be greater in mature female cows.

### Example 10

This example illustrates the effect of repeated blood draws on the proliferative capacity of the platelet-rich plasma (PRP) isolated from young and mature cows of both sexes. PRP was isolated from the cows listed in Table 1 over a 3-month period. PRP samples isolated at the beginning, middle, and end of the 3-month time period were evaluated for their ability to stimulate proliferation of bovine myoblasts. At each respective time point, bovine myoblasts were isolated from fresh muscle biopsies, dissociated from the tissue, resuspended in DMEM supplemented with 20% fetal bovine serum, and seeded on 24-well plates at a loading density of 25,000 cells/well. After 4 hours, the cell media was removed, and the cell incubated with either 10% PRP or 10% FBS in DMEM (plus 1% P/S+Normocin and Heparin). After 72 hours, the cell number for each well was counted using counting slides and degree of cell proliferation determined (data was normalized to the FBS treated groups). The results in this example suggested the following: (1) the proliferative capacity of PRP does not decrease with repeated collection over a 3-month period, (2) repeated collection of PRP increases its proliferative capacity in mature cohorts, (3) weekly collection of PRP enhanced bovine muscle proliferation better than biweekly collections, and (4) the cow's gender does not have a significant effect on the ability of PRP to promote cell proliferation.

Numerous modifications and variations in the practice of the invention are expected to occur to those skilled in the art upon consideration of the foregoing description on the presently preferred embodiments thereof. Consequently, the only limitations which should be placed upon the scope of the present invention are those that appear in the appended claims.

In addition, while several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teaching of the present disclosure is/are used. Those skilled in the art will recognize or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A composition, comprising:
cell culture media comprising a platelet lysate arising from a non-human animal, wherein the platelet lysate is present at 2% to 20% by weight of the cell culture media.

2-122. (canceled)

123. The composition of claim 1, wherein the platelet lysate is present at 5% to 15% by weight of the cell culture media.

124-127. (canceled)

128. The composition of claim 1, wherein the non-human animal is selected from the group consisting of cow, sheep, goat, swine, deer, camel, whale, fowl, fish, crab, and shrimp.

129. The cell culture media of claim 1, further comprising a growth factor.

130. The cell culture media of claim 129, wherein the growth factor is basic fibroblast growth factor.

131. The cell culture media of claim 130, wherein the concentration of basic fibroblast growth factor is at least 2 ng/mL.

132. A method of raising non-human cells for cell-based products in culture comprising:
exposing a plurality of non-human animal cells in a bioreactor to a cell culture media comprising a platelet lysate arising from a non-human animal, wherein the platelet lysate is present at 2% to 20% by weight of the cell culture media.

133. (canceled)

134. The method of claim 132, wherein the non-human animal cell is a cow.

135. The method of claim 132, wherein the non-human animal cell is a sheep.

136. The method of claim 132, wherein the non-human animal cell is a goat.

137. The method of claim 132, wherein the non-human animal cell is a swine.

138. The method of claim 132, wherein the non-human animal cell arise from is a camel.

139. The method of claim 132, wherein the non-human animal cell is a fish.

140. A method, comprising:
adding cell culture media comprising a platelet lysate arising from a non-human animal to a bioreactor, wherein the platelet lysate is present at 2% to 20% by weight of the cell culture media;
adding non-human animal cells to the bioreactor; and
growing a cell-based meat product from the non-human animal cells in the bioreactor.

141. The composition of claim 1, wherein the composition further comprises a plurality of microcarrier scaffolds.

142. The composition of claim 141, wherein the plurality of microcarrier scaffolds have an average diameter of less than 1 mm.

143. The composition of claim 1, wherein the cell culture media comprises DMEM.

144. The method of claim 132, wherein the composition further comprises a plurality of microcarrier scaffolds.

145. The method of claim 144, wherein the plurality of microcarrier scaffolds have an average diameter of less than 1 mm.

146. The method of claim 144, wherein the non-human animal cells and the platelet lysate arising from a non-human animal are from the same animal species.

147. The method of claim 132, wherein the cell culture media comprises DMEM.

148. The method of claim 140, comprising adding a plurality of microcarrier scaffolds to the bioreactor.

149. The method of claim 148, wherein the plurality of microcarrier scaffolds have an average diameter of less than 1 mm.

150. The method of claim 140, wherein the non-human animal cells and the platelet lysate arising from a non-human animal are from the same animal species.

151. The method of claim 140, wherein the cell culture media comprises DMEM.
